(19) United States
(12) Patent Application Publication
(10) Pub. No.: US 2021/0333288 A1
(43) Pub. Date: Oct. 28, 2021

(54) GLYCAN OXONIUM ION PROFILING OF GLYCOSYLATED PROTEINS

(71) Applicant: Momenta Pharmaceuticals, Inc., Cambridge, MA (US)

(72) Inventors: James Madsen, Medford, MA (US); Victor Farutin, Watertown, MA (US)

(21) Appl. No.: 16/326,017

(22) PCT Filed: Aug. 17, 2017
(86) PCT No.: PCT/US17/47395

§ 371 Date: Feb. 15, 2019

Related U.S. Application Data
(60) Provisional application No. 62/376,072, filed on Aug. 17, 2016.

(51) Int. Cl. G01N 33/68

(52) U.S. Cl. CPC G01N 33/6848; G01N 2440/38; G01N 33/6854

(57) ABSTRACT

Methods disclosed herein include an improved technique for comparing a glycosylation profile of a first protein (e.g., an innovator protein drug) with a glycosylation profile of a second protein (e.g., a corresponding biogeneric/biosimilar). For example, a method of manufacture can include providing or obtaining a batch of a test glycoprotein drug substance, using mass spectrometry to acquire a test oxonium ion profile from a sample of the test glycoprotein drug substance batch, comparing the test oxonium ion profile to a corresponding target oxonium ion profile of a target glycoprotein drug product, and processing the batch of the test glycoprotein drug substance as a drug product if the difference between the test oxonium ion profile and the corresponding target oxonium ion profile is tolerable, or taking an alternative action if the difference between the test oxonium ion profile and the target oxonium ion profile is not tolerable.

### FIELD OF THE INVENTION

This disclosure provides methods related to glycan oxonium profiling of glycosylated proteins.

### BACKGROUND

Therapeutic polypeptides are an important class of therapeutic biotechnology products. Therapeutic antibodies account for a majority of therapeutic biologic products. Fusion proteins also represent a significant portion of therapeutic biologic products. Therapeutic biologic products, such as antibodies and fusion proteins, can be glycosylated and can contain several sites along the protein backbone where heterogeneous glycan moieties reside. As protein glycosylation can play an important role in a variety of cellular functions, understanding the glycosylation of therapeutic biologic products can be beneficial.

### SUMMARY

The present disclosure provides, in part, methods for evaluating, identifying, analyzing and/or producing (e.g., manufacturing) a protein, e.g., a glycoprotein, e.g., an antibody and/or a biosimilar antibody. In some instances, methods described herein allow for high resolution evaluation of a protein (e.g., a glycoprotein, e.g., an antibody) useful for, inter alia, manufacturing and/or evaluating a protein, such as a biosimilar protein, e.g., a biosimilar antibody.

In one aspect, the disclosure provides methods of analyzing a protein (e.g., a protein preparation (e.g., a batch of a protein preparation), a protein drug substance (e.g., a batch of a protein drug substance), or a protein drug product (e.g., a batch of a protein drug product)). In some embodiments, a method includes using mass spectrometry to acquire (e.g., detect, measure, determine, receive, or obtain) an oxonium ion profile of a sample of a protein (e.g., a protein preparation (e.g., a batch of a protein preparation), a protein drug substance (e.g., a batch of a protein drug substance), or a protein drug product (e.g., a batch of a protein drug product)).

In some embodiments, using mass spectrometry includes performing liquid chromatography-tandem mass spectrometry. In some embodiments, using mass spectrometry includes digesting a sample of a protein (e.g., a protein preparation (e.g., a batch of a protein preparation), a protein drug substance (e.g., a batch of a protein drug substance), or a protein drug product (e.g., a batch of a protein drug product)) to produce a plurality of glycopeptides and/or glycans. In some embodiments, using mass spectrometry includes performing data-independent mass spectrometry. In some embodiments, using mass spectrometry includes performing all ion fragmentation.

In some embodiments, an oxonium ion profile includes one or more MS signals associated with one or more oxonium ion-containing fragments. Oxonium ion-containing fragments can include, for example, a HexNAc internal fragment, a fragment containing a Hex, a fragment containing a HexNAc, a fragment containing a sialic acid±H2O, a fragment containing a Hex+HexNAc, or combinations thereof. In some embodiments, an oxonium ion profile includes one or more MS signals associated with levels of oxonium ion-containing fragments that include the following oxonium ions: (i) HexNAc internal fragment; (ii) Hex; (iii) HexNAc; (iv) sialic acid; (v) sialic acid+H₂O; (vi) Hex+HexNAc; or (vii) combinations thereof. In some embodiments, an oxonium ion profile includes one or more MS signals associated with levels of oxonium ion-containing fragments that include oxonium ions having the following m/z values at: (i) m/z 138.06 (monoisotopic), 139.06 (+1 isotope), 140.06 (+2 isotope); (ii) m/z 163.06 (monoisotopic), 164.06 (+1 isotope), 165.06 (+2 isotope); (iii) m/z 204.09 (monoisotopic), 205.09 (+1 isotope), 206.09 (+2 isotope); (iv) m/z 274.09 (monoisotopic), 275.10 (+1 isotope), 276.10 (+2 isotope); (v) m/z 292.10 (monoisotopic), 293.11 (+1 isotope), 294.11 (+2 isotope); (vi) m/z 366.14 (monoisotopic), 367.14 (+1 isotope), 368.14 (+2 isotope); or (vii) combinations thereof. In some instances, an oxonium ion includes an isotope, for example, D, C¹³, N¹⁵, or O¹⁸. In some embodiments, an oxonium ion profile includes one or more MS signals associated with levels of oxonium ion-containing fragments that include oxonium ions within the following m/z ranges: (i) m/z 138.05-138.07; (ii) m/z 163.05-163.07; (iii) m/z 204.08-204.10; (iv) m/z 274.08-274.10; (v) m/z 292.09-292.11; (vi) m/z 366.13-366.15; or (vii) combinations thereof.

In some embodiments, a method of analysis includes providing (e.g., producing or expressing (e.g., in small scale or large scale cell culture) or manufacturing) a protein (e.g., a protein preparation (e.g., a batch of a protein preparation), a protein drug substance (e.g., a batch of a protein drug substance), or a protein drug product (e.g., a batch of a protein drug product)). In some embodiments, a method of analysis includes obtaining (e.g., receiving and/or purchasing from a third party (including a contractually related third party or a non-contractually-related (e.g., an independent) third party) a protein (e.g., a protein preparation (e.g., a batch of a protein preparation), a protein drug substance (e.g., a batch of a protein drug substance), or a protein drug product (e.g., a batch of a protein drug product)).

In some embodiments, a protein is a glycoprotein. In some embodiments, a glycoprotein is or includes an antibody or an antibody fragment, e.g., a Fab fragment and/or an Fc fragment. In some embodiments, a glycoprotein is or includes an Fc fusion protein, or a fragment thereof. In some embodiments, a glycoprotein is or includes abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and/or ustekinumab (Stelara®, Janssen).

In another aspect, the disclosure provides methods of manufacture. In some embodiments, a method includes providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a first protein (e.g., a first protein preparation (e.g., a batch of a first protein preparation), a first protein drug substance (e.g., a batch of a first protein drug substance), or a first protein drug product (e.g., a batch of a first protein drug product)); using mass spectrometry to acquire (e.g., detect, measure, determine, receive, or obtain) a first oxonium ion profile from a sample of the first protein; and comparing the first oxonium ion profile to a corresponding second oxonium ion profile of a second protein (e.g., a second protein preparation (e.g., a batch of a second protein preparation), a second protein drug substance (e.g., a batch of a second protein drug substance), or a second protein drug product (e.g., a batch of a second protein drug product)).

In some embodiments, a method includes determining whether a difference between a first oxonium ion profile and a corresponding second oxonium ion profile is tolerable. In some embodiments, a first oxonium ion profile includes one or more MS signals that are each associated with a level of an oxonium ion-containing fragment. In some embodiments, a corresponding second oxonium ion profile includes one or more MS signals that are each associated with a level of an oxonium ion-containing fragment. In some embodiments, a difference between a first oxonium ion profile and a corresponding second oxonium ion profile is tolerable if the difference between a level of at least one oxonium ion-containing fragment derived from the first oxonium ion profile and a level of at least one oxonium ion-containing fragment derived from the corresponding second oxonium ion profile is less than a predetermined value. In some embodiments, a difference between a first oxonium ion profile and a corresponding second oxonium ion profile is not tolerable if the difference between a level of at least one oxonium ion-containing fragment derived from the first oxonium ion profile and a level of at least one oxonium ion-containing fragment derived from the corresponding second oxonium ion profile is greater than a predetermined value. In some embodiments, a difference between a first oxonium ion profile and a corresponding second oxonium ion profile is tolerable if the difference between 2, 3, 4, 5, 6, 7, 8, 9, 10 or more MS signals of the first oxonium ion profile and 2, 3, 4, 5, 6, 7, 8, 9, 10 or more corresponding MS signals of the second oxonium ion profile is each less than a predetermined value. In some embodiments, a difference between a first oxonium ion profile and a corresponding second oxonium ion profile is not tolerable if the difference between 1, 2, 3, 4, 5, 6, 7, 8, 9, 10 or more MS signals of the first oxonium ion profile and 1, 2, 3, 4, 5, 6, 7, 8, 9, 10 or more corresponding MS signals of the second oxonium ion profile is each greater than a predetermined value.

In some embodiments, a predetermined value is equivalent to the variability in oxonium ion profiles determined for three, four, five, ten or more distinct batches of a first protein (e.g., a test protein, e.g., a test glycoprotein) or second protein (e.g., a target protein, e.g., a target glycoprotein). In some embodiments, a predetermined value is 20%, 15%, 10% or 5%, e.g., of a level of at least one oxonium ion-containing fragment derived from the first oxonium ion profile or the second oxonium ion profile. In some embodiments, a predetermined value is 20%, 15%, 10% or 5%, e.g., of a respective MS signal of the first oxonium ion profile or the second oxonium ion profile.

In some embodiments, a method includes processing the batch of the test glycoprotein drug substance as a drug product if the difference between the test oxonium ion profile and the corresponding target oxonium ion profile is tolerable. In some embodiments, processing includes formulating a batch of a test glycoprotein drug substance; combining a batch of a test glycoprotein drug substance with a second component, e.g., an excipient or buffer; changing a concentration of a batch of a test glycoprotein drug substance in a drug product; lyophilizing a batch of a test glycoprotein drug substance; combining a first and a second aliquot of a batch of a test glycoprotein drug substance to provide a third, larger aliquot; dividing a batch of a test glycoprotein drug substance into smaller aliquots; disposing a batch of a test glycoprotein drug substance into a container, e.g., a gas or liquid tight container; packaging a batch of a test glycoprotein drug substance; associating a container comprising a batch of a test glycoprotein drug substance with a label (e.g., labeling); shipping or moving a batch of a test glycoprotein drug substance to a different location; or a combination thereof.

In some embodiments, a method includes taking an alternative action if the difference between the test oxonium ion profile and the target oxonium ion profile is not tolerable. In some embodiments, an alternative action includes disposing of a batch of a test glycoprotein drug substance, classifying for disposal a batch of a test glycoprotein drug substance, labeling a batch of a test glycoprotein drug substance for disposal, reprocessing a batch of a test glycoprotein drug substance or a combination thereof.

In some embodiments, using mass spectrometry includes performing liquid chromatography-tandem mass spectrometry. In some embodiments, using mass spectrometry includes digesting a sample of a first protein (e.g., a first protein preparation (e.g., a batch of a first protein preparation) or a first protein drug substance (e.g., a batch of a first protein drug substance)) to produce a plurality of glycopeptides and/or glycans. In some embodiments, using mass spectrometry includes performing data-independent mass spectrometry. In some embodiments, using mass spectrometry includes performing all ion fragmentation.

In some embodiments, an oxonium ion profile includes one or more MS signals associated with one or more oxonium ion-containing fragments. Oxonium ion-containing fragments can include, for example, a HexNAc internal fragment, a fragment containing a Hex, a fragment containing a HexNAc, a fragment containing a sialic acid±H₂O, a fragment containing a Hex+HexNAc, or combinations thereof. In some embodiments, an oxonium ion profile includes one or more MS signals associated with levels of oxonium ion-containing fragments that include the following oxonium ions: (i) HexNAc internal fragment; (ii) Hex; (iii) HexNAc; (iv) sialic acid; (v) sialic acid+H₂O; (vi) Hex+HexNAc; or (vii) combinations thereof. In some embodiments, an oxonium ion profile includes one or more MS signals associated with levels of oxonium ion-containing fragments that include oxonium ions having the following m/z values at: (i) m/z 138.06 (monoisotopic), 139.06 (+1 isotope), 140.06 (+2 isotope); (ii) m/z 163.06 (monoisotopic), 164.06 (+1 isotope), 165.06 (+2 isotope); (iii) m/z 204.09 (monoisotopic), 205.09 (+1 isotope), 206.09 (+2 isotope); (iv) m/z 274.09 (monoisotopic), 275.10 (+1 isotope), 276.10 (+2 isotope); (v) m/z 292.10 (monoisotopic), 293.11 (+1 isotope), 294.11 (+2 isotope); (vi) m/z 366.14 (monoisotopic), 367.14 (+1 isotope), 368.14 (+2 isotope); or (vii) combinations thereof. In some instances, an oxonium ion includes an isotope, for example, D, C¹³, N¹⁵, or O¹⁸. In some embodiments, an oxonium ion profile includes one or more MS signals associated with levels of oxonium ion-containing fragments that include oxonium ions within the following m/z ranges: (i) m/z 138.05-138.07; (ii) m/z 163.05-163.07; (iii) m/z 204.08-204.10; (iv) m/z 274.08-274.10; (v) m/z 292.09-292.11; (vi) m/z 366.13-366.15; or (vii) combinations thereof.

In some embodiments, a method includes producing a representation of a comparison of a first oxonium ion profile to a corresponding second oxonium ion profile of a second protein. In some embodiments, comparing the first oxonium ion profile to a corresponding second oxonium ion profile of a second protein is performed visually. In some embodiments, comparing the first oxonium ion profile to a corresponding second oxonium ion profile of a second protein includes binning data (e.g., based on retention time or mass-to-charge ratio) and/or comparing binned data.

In some embodiments, a method includes using mass spectrometry to acquire a second oxonium ion profile of a second protein. In some embodiments, using mass spectrometry includes performing liquid chromatography-tandem mass spectrometry. In some embodiments, using mass spectrometry includes digesting a sample of a second protein (e.g., a second protein preparation (e.g., a batch of a second protein preparation) or a second protein drug substance (e.g., a batch of a second protein drug substance)) to produce a plurality of glycopeptides and/or glycans. In some embodiments, using mass spectrometry includes performing data-independent mass spectrometry. In some embodiments, using mass spectrometry includes performing all ion fragmentation.

In some embodiments, a first protein and/or a second protein is or includes a glycoprotein. In some embodiments, a glycoprotein is or includes an antibody or an antibody fragment, e.g., a Fab fragment and/or an Fc fragments. In some embodiments, a first protein is a test protein and a second protein is a target protein. In some embodiments, a first protein is approved under a secondary approval process. In some embodiments, a second protein is approved under a primary approval process (e.g., under a BLA).

In some embodiments, a first protein and/or a second protein is or includes abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and/or ustekinumab (Stelara®, Janssen).

In some embodiments, a first protein has an amino acid sequence having at least 90%, at least 95%, at least 98%, or at least 99% sequence identity to an amino acid sequence of a second protein. In some embodiments, a first protein has an amino acid sequence that is identical to an amino acid sequence of a second protein. In some embodiments, a first protein has an amino acid sequence that differs from an amino acid sequence of a second protein (e.g., due to mutation, addition, or deletion) by 10, 9, 8, 7, 6, 5, 4, 3, 2, or 1 amino acid at the C-terminal end or the N-terminal end of the second protein.

In another aspect, the disclosure features a method of manufacture. In some embodiments, a method includes providing (e.g., producing or expressing (e.g., in small scale or large scale cell culture) or manufacturing) or obtaining (e.g., receiving and/or purchasing from a third party (including a contractually related third party or a non-contractually-related (e.g., an independent) third party) a test glycoprotein drug substance; using mass spectrometry to acquire (e.g., detect, measure, determine, receive, or obtain) a test oxonium ion profile from a sample of the test glycoprotein drug substance batch; comparing the test oxonium ion profile to a corresponding target oxonium ion profile of a target glycoprotein drug product; and processing the batch of the test glycoprotein drug substance as a drug product if the difference between the test oxonium ion profile and the corresponding target oxonium ion profile is tolerable; or taking an alternative action if the difference between the test oxonium ion profile and the target oxonium ion profile is not tolerable.

In some embodiments, a method includes determining whether a difference between a test oxonium ion profile and a corresponding target oxonium ion profile is tolerable. In some embodiments, a test oxonium ion profile includes one or more MS signals that are each associated with a level of an oxonium ion-containing fragment. In some embodiments, a corresponding target oxonium ion profile includes one or more MS signals that are each associated with a level of an oxonium ion-containing fragment. In some embodiments, a difference between a test oxonium ion profile and a corresponding target oxonium ion profile is tolerable if the difference between a level of at least one oxonium ion-containing fragment derived from the test oxonium ion profile and a level of at least one oxonium ion-containing fragment derived from the corresponding target oxonium ion profile is less than a predetermined value. In some embodiments, a difference between a test oxonium ion profile and a corresponding target oxonium ion profile is not tolerable if the difference between a level of at least one oxonium ion-containing fragment derived from the test oxonium ion profile and a level of at least one oxonium ion-containing fragment derived from the corresponding target oxonium ion profile is greater than a predetermined value. In some embodiments, a difference between a test oxonium ion profile and a corresponding target oxonium ion profile is tolerable if the difference between 2, 3, 4, 5, 6, 7, 8, 9, 10 or more MS signals of the test oxonium ion profile and 2, 3, 4, 5, 6, 7, 8, 9, 10 or more corresponding MS signals of the target oxonium ion profile is each less than a predetermined value. In some embodiments, a difference between a test oxonium ion profile and a corresponding target oxonium ion profile is not tolerable if the difference between 1, 2, 3, 4, 5, 6, 7, 8, 9, 10 or more MS signals of the test oxonium ion profile and 1, 2, 3, 4, 5, 6, 7, 8, 9, 10 or more corresponding MS signals of the target oxonium ion profile is each greater than a predetermined value.

In some embodiments, a predetermined value is equivalent to the variability in oxonium ion profiles determined for three, four, five, ten or more distinct batches of a target glycoprotein drug product. In some embodiments, a predetermined value is 20%, 15%, 10% or 5%, e.g., of a level of at least one oxonium ion-containing fragment derived from the test oxonium ion profile or the target oxonium ion profile. In some embodiments, a predetermined value is 20%, 15%, 10% or 5%, e.g., of a respective MS signal of the test oxonium ion profile or the target oxonium ion profile.

In some embodiments, processing includes formulating a batch of a test glycoprotein drug substance; combining a batch of a test glycoprotein drug substance with a second component, e.g., an excipient or buffer; changing a concentration of a batch of a test glycoprotein drug substance in a drug product; lyophilizing a batch of a test glycoprotein drug substance; combining a first and a second aliquot of a batch of a test glycoprotein drug substance to provide a third, larger aliquot; dividing a batch of a test glycoprotein drug substance into smaller aliquots; disposing a batch of a test glycoprotein drug substance into a container, e.g., a gas or liquid tight container; packaging a batch of a test glycoprotein drug substance; associating a container comprising a batch of a test glycoprotein drug substance with a label (e.g., labeling); shipping or moving a batch of a test glycoprotein drug substance to a different location; or a combination thereof.

In some embodiments, taking an alternative action includes disposing of a batch of a test glycoprotein drug substance, classifying for disposal a batch of a test glycoprotein drug substance, labeling a batch of a test glycoprotein drug substance for disposal, reprocessing a batch of a test glycoprotein drug substance or a combination thereof.

In some embodiments, using mass spectrometry includes performing liquid chromatography-tandem mass spectrometry. In some embodiments, using mass spectrometry includes digesting a sample of a test protein drug substance (e.g., a batch of a test protein drug substance)) to produce a plurality of glycopeptides and/or glycans. In some embodiments, using mass spectrometry includes performing data-independent mass spectrometry. In some embodiments, using mass spectrometry includes performing all ion fragmentation.

In some embodiments, an oxonium ion profile includes one or more MS signals associated with one or more oxonium ion-containing fragments. Oxonium ion-containing fragments can include, for example, a HexNAc internal fragment, a fragment containing a Hex, a fragment containing a HexNAc, a fragment containing a sialic acid±H₂O, a fragment containing a Hex+HexNAc, or combinations thereof. In some embodiments, an oxonium ion profile includes one or more MS signals associated with levels of oxonium ion-containing fragments that include the following oxonium ions: (i) HexNAc internal fragment; (ii) Hex; (iii) HexNAc; (iv) sialic acid; (v) sialic acid+H₂O; (vi) Hex+HexNAc; or (vii) combinations thereof. In some embodiments, an oxonium ion profile includes one or more MS signals associated with levels of oxonium ion-containing fragments that include oxonium ions having the following m/z values at: (i) m/z 138.06 (monoisotopic), 139.06 (+1 isotope), 140.06 (+2 isotope); (ii) m/z 163.06 (monoisotopic), 164.06 (+1 isotope), 165.06 (+2 isotope); (iii) m/z 204.09 (monoisotopic), 205.09 (+1 isotope), 206.09 (+2 isotope); (iv) m/z 274.09 (monoisotopic), 275.10 (+1 isotope), 276.10 (+2 isotope); (v) m/z 292.10 (monoisotopic), 293.11 (+1 isotope), 294.11 (+2 isotope); (vi) m/z 366.14 (monoisotopic), 367.14 (+1 isotope), 368.14 (+2 isotope); or (vii) combinations thereof. In some instances, an oxonium ion includes an isotope, for example, D, C¹³, N¹⁵, or O¹⁸. In some embodiments, an oxonium ion profile includes one or more MS signals associated with levels of oxonium ion-containing fragments that include oxonium ions within the following m/z ranges: (i) m/z 138.05-138.07; (ii) m/z 163.05-163.07; (iii) m/z 204.08-204.10; (iv) m/z 274.08-274.10; (v) m/z 292.09-292.11; (vi) m/z 366.13-366.15; or (vii) combinations thereof.

In some embodiments, a method includes producing a representation of the comparison between a test oxonium ion profile and a corresponding target oxonium ion profile. In some embodiments, comparing is performed visually. In some embodiments, comparing includes binning data (e.g., based on retention time or mass-to-charge ratio).

In some embodiments, a method includes using mass spectrometry to acquire a target oxonium ion profile of a target glycoprotein drug product. In some embodiments, using mass spectrometry includes performing liquid chromatography-tandem mass spectrometry. In some embodiments, using mass spectrometry includes digesting a sample of a target glycoprotein drug product (e.g., a batch of a target protein drug product)) to produce a plurality of glycopeptides and/or glycans. In some embodiments, using mass spectrometry includes performing data-independent mass spectrometry. In some embodiments, using mass spectrometry includes performing all ion fragmentation.

In some embodiments, a test glycoprotein drug substance and/or target glycoprotein drug product is or includes an antibody or an antibody fragment, e.g., a Fab fragment and/or an Fc fragment. In some embodiments, a target glycoprotein drug product is approved under a primary approval process (e.g., under a BLA). In some embodiments, a test glycoprotein drug substance and/or target glycoprotein drug product is or includes abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and/or ustekinumab (Stelara®, Janssen).

In some embodiments, a test glycoprotein drug substance has an amino acid sequence that is at least 90%, at least 95%, at least 98%, or at least 99% identical to an amino acid sequence of a target glycoprotein drug product (e.g., a glycoprotein included in a target glycoprotein drug product). In some embodiments, a test glycoprotein drug substance has an amino acid sequence that is identical to an amino acid sequence of a target glycoprotein drug product (e.g., a glycoprotein included in a target glycoprotein drug product). In some embodiments, a test glycoprotein drug substance has an amino acid sequence that differs from an amino acid sequence of a target glycoprotein drug product (e.g., a glycoprotein included in a target glycoprotein drug product) (e.g., due to mutation, addition, or deletion) by 10, 9, 8, 7, 6, 5, 4, 3, 2, or 1 amino acid at the C-terminal end or the N-terminal end of the target glycoprotein drug product.

In another aspect, the disclosure features methods of manufacture. In some embodiments, a method of manufacture includes comparing a test oxonium ion profile from a sample of a batch of a test glycoprotein drug substance to a corresponding target oxonium ion profile of a target glycoprotein drug product. In some embodiments, a method includes processing the batch of the test glycoprotein drug substance as a drug product if the difference between the test oxonium ion profile and the corresponding target oxonium ion profile is tolerable; or taking an alternative action if the difference between the test oxonium ion profile and the target oxonium ion profile is not tolerable.

In some embodiments, a difference between a test oxonium ion profile and a corresponding target oxonium ion profile is tolerable if the difference between a level of at least one oxonium ion-containing fragment derived from the test oxonium ion profile and a level of at least one oxonium ion-containing fragment derived from the corresponding target oxonium ion profile is less than a predetermined value. In some embodiments, a difference between a test oxonium ion profile and a corresponding target oxonium ion profile is not tolerable if the difference between a level of at least one oxonium ion-containing fragment derived from the test oxonium ion profile and a level of at least one oxonium ion-containing fragment derived from the corresponding target oxonium ion profile is greater than a predetermined value. In some embodiments, a difference between a test oxonium ion profile and a corresponding target oxonium ion profile is tolerable if the difference between 2, 3, 4, 5, 6, 7, 8, 9, 10 or more MS signals of the test oxonium ion profile and 2, 3, 4, 5, 6, 7, 8, 9, 10 or more corresponding MS signals of the corresponding target oxonium ion profile is each less than a predetermined value. In some embodiments, a difference between a test oxonium ion profile and a corresponding target oxonium ion profile is not tolerable if the difference between 1, 2, 3, 4, 5, 6, 7, 8, 9, 10 or more MS signals of the test oxonium ion profile and 1, 2, 3, 4, 5, 6, 7, 8, 9, 10 or more corresponding MS signals of the corresponding target oxonium ion profile is each greater than a predetermined value.

In some embodiments, a predetermined value is equivalent to the variability in oxonium ion profiles determined for three, four, five, ten or more distinct batches of a test glycoprotein drug substance or a target glycoprotein drug product. In some embodiments, a predetermined value is 20%, 15%, 10% or 5%, e.g., of a level of at least one oxonium ion-containing fragment derived from a test oxonium ion profile or a corresponding target oxonium ion profile. In some embodiments, a predetermined value is 20%, 15%, 10% or 5%, e.g., of a respective MS signal of a test oxonium ion profile or a corresponding target oxonium ion profile.

In some embodiments, processing includes formulating a batch of a test glycoprotein drug substance; combining a batch of a test glycoprotein drug substance with a second component, e.g., an excipient or buffer; changing a concentration of a batch of a test glycoprotein drug substance in a drug product; lyophilizing a batch of a test glycoprotein drug substance; combining a first and a second aliquot of a batch of a test glycoprotein drug substance to provide a third, larger aliquot; dividing a batch of a test glycoprotein drug substance into smaller aliquots; disposing a batch of a test glycoprotein drug substance into a container, e.g., a gas or liquid tight container; packaging a batch of a test glycoprotein drug substance; associating a container comprising a batch of a test glycoprotein drug substance with a label (e.g., labeling); shipping or moving a batch of a test glycoprotein drug substance to a different location; or a combination thereof.

In some embodiments, taking an alternative action includes disposing of a batch of a test glycoprotein drug substance, classifying for disposal a batch of a test glycoprotein drug substance, labeling a batch of a test glycoprotein drug substance for disposal, reprocessing a batch of a test glycoprotein drug substance or a combination thereof.

In some embodiments, a test oxonium ion profile and/or a target oxonium ion profile is acquired using mass spectrometry. In some embodiments, a test oxonium ion profile and/or a target oxonium ion profile is indirectly acquired (e.g., acquired from a third party).

In some embodiments, using mass spectrometry includes performing liquid chromatography-tandem mass spectrometry. In some embodiments, using mass spectrometry includes digesting a sample of a test glycoprotein drug substance (e.g., a batch of a test glycoprotein drug substance) and/or digesting a sample of a target glycoprotein drug product (e.g., a batch of a target glycoprotein drug product) to produce a plurality of glycopeptides and/or glycans. In some embodiments, using mass spectrometry includes performing data-independent mass spectrometry. In some embodiments, using mass spectrometry includes performing all ion fragmentation.

In some embodiments, an oxonium ion profile includes one or more MS signals associated with one or more oxonium ion-containing fragments. Oxonium ion-containing fragments can include, for example, a HexNAc internal fragment, a fragment containing a Hex, a fragment containing a HexNAc, a fragment containing a sialic acid±H2O, a fragment containing a Hex+HexNAc, or combinations thereof. In some embodiments, an oxonium ion profile includes one or more MS signals associated with levels of oxonium ion-containing fragments that include the following oxonium ions: (i) HexNAc internal fragment; (ii) Hex; (iii) HexNAc; (iv) sialic acid; (v) sialic acid+H₂O; (vi) Hex+HexNAc; or (vii) combinations thereof. In some embodiments, an oxonium ion profile includes one or more MS signals associated with levels of oxonium ion-containing fragments that include oxonium ions having the following m/z values at: (i) m/z 138.06 (monoisotopic), 139.06 (+1 isotope), 140.06 (+2 isotope); (ii) m/z 163.06 (monoisotopic), 164.06 (+1 isotope), 165.06 (+2 isotope); (iii) m/z 204.09 (monoisotopic), 205.09 (+1 isotope), 206.09 (+2 isotope); (iv) m/z 274.09 (monoisotopic), 275.10 (+1 isotope), 276.10 (+2 isotope); (v) m/z 292.10 (monoisotopic), 293.11 (+1 isotope), 294.11 (+2 isotope); (vi) m/z 366.14 (monoisotopic), 367.14 (+1 isotope), 368.14 (+2 isotope); or (vii) combinations thereof. In some instances, an oxonium ion includes an isotope, for example, D, C¹³, N¹⁵, or O¹⁸. In some embodiments, an oxonium ion profile includes one or more MS signals associated with levels of oxonium ion-containing fragments that include oxonium ions within the following m/z ranges: (i) m/z 138.05-138.07; (ii) m/z 163.05-163.07; (iii) m/z 204.08-204.10; (iv) m/z 274.08-274.10; (v) m/z 292.09-292.11; (vi) m/z 366.13-366.15; or (vii) combinations thereof.

In some embodiments, a test oxonium ion profile includes one or more MS signals that are each associated with a level of an oxonium ion-containing fragment. In some embodiments, a corresponding target oxonium ion profile includes one or more MS signals that are each associated with a level of an oxonium ion-containing fragment.

In some embodiments, a method includes producing a representation of the comparison between a test oxonium ion profile and a corresponding target oxonium ion profile. In some embodiments, comparing is performed visually. In some embodiments, comparing includes binning data (e.g., based on retention time or mass-to-charge ratio).

In some embodiments, a test glycoprotein drug substance and/or a target glycoprotein drug product is or includes an antibody or an antibody fragment, e.g., a Fab fragment and/or an Fc fragment. In some embodiments, a target glycoprotein drug product is approved under a primary approval process (e.g., under a BLA). In some embodiments, a test glycoprotein drug substance and/or a target glycoprotein drug product is or includes abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and/or ustekinumab (Stelara®, Janssen).

In some embodiments, a test glycoprotein drug substance has an amino acid sequence that is at least 90%, at least 95%, at least 98%, or at least 99% identical to an amino acid sequence of a target glycoprotein drug product (e.g., a glycoprotein included in a target glycoprotein drug product). In some embodiments, a test glycoprotein drug substance has an amino acid sequence that is identical to an amino acid sequence of a target glycoprotein drug product (e.g., a glycoprotein included in a target glycoprotein drug product). In some embodiments, a test glycoprotein drug substance has an amino acid sequence that differs from an amino acid sequence of a target glycoprotein drug product (e.g., a glycoprotein included in a target glycoprotein drug product) (e.g., due to mutation, addition, or deletion) by 10, 9, 8, 7, 6, 5, 4, 3, 2, or 1 amino acid at the C-terminal end or the N-terminal end of the target glycoprotein drug product.

In any of the aspects described herein, methods can further include, e.g., one or more of: memorializing a comparison and/or results of a comparison (e.g., between a test MS signal and a target MS signal) using a recordable medium (e.g., on paper or in a computer readable medium, e.g., in a Certificate of Testing, Material Safety Data Sheet (MSDS), batch record, or Certificate of Analysis (CofA)); informing a party or entity (e.g., a contractual or manufacturing partner, a care giver or other end-user, a regulatory entity, e.g., the FDA or other U.S., European, Japanese, Chinese or other governmental agency, or another entity, e.g., a compendial entity (e.g., U.S. Pharmacopoeia (USP)) or insurance company) of the comparison and/or results of the comparison.

### Definitions

In general, a “protein,” as used herein, is a polypeptide (i.e., a string of at least two amino acids linked to one another by peptide bonds). Proteins, polypeptides and peptides may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “peptide” as used herein refers to a polypeptide that is typically relatively short, for example having a length of less than about 100 amino acids, less than about 50 amino acids, less than 20 amino acids, or less than 10 amino acids.

As used herein, a “glycoprotein” refers to an amino acid sequence that includes one or more oligosaccharide chains (e.g., glycans) covalently attached thereto. Exemplary amino acid sequences include peptides, polypeptides, proteins and fusion proteins (including fusion proteins including antibodies or antibody fragments). Exemplary glycoproteins can include glycosylated antibodies, antibody-like molecules (e.g., Fc fusion proteins) or fragments thereof. An antibody can be a murine, chimeric, reverse-chimeric, humanized, or human antibody. Exemplary antibodies may include monoclonal antibodies and/or fragments thereof, polyclonal antibodies and/or fragments thereof, and Fc domain containing fusion proteins (e.g., fusion proteins containing an Fc region of IgG1, or a glycosylated portion thereof).

The term “protein preparation” as used herein refers to a mixture of proteins obtained according to a particular production method. Proteins in a protein preparation may be the same or different, i.e., a protein preparation may include several copies of the same protein and/or a mixture of different proteins. In some embodiments, a production method can include a recombinant preparation step using cultured cells that have been engineered to express one or more proteins in a protein preparation (or to express proteins at a relevant level or under relevant conditions). In some embodiments, a production method may further include an isolation step in which proteins are isolated from certain components of engineered cells (e.g., by lysing cells and pelleting a protein component by centrifugation). In some embodiments, a production method may also include a purification step in which proteins in a protein preparation are separated (e.g., by chromatography) from other cellular components, e.g., other proteins or organic components that were used in earlier steps. It will be appreciated that these steps are non-limiting and that any number of additional productions steps may be included. Different protein preparations may be prepared by the same production method but on different occasions (e.g., different batches). Alternatively, different protein preparations may be prepared by different production methods. Two production methods may differ in any way (e.g., expression vector, engineered cell type, culture conditions, isolation procedure, purification conditions, etc.).

As used herein, a “glycoprotein preparation” is a composition or mixture that includes at least one glycoprotein. In some instances, a glycoprotein preparation (e.g., such as a glycoprotein drug substance or a precursor thereof) can be a sample from a proposed or test batch of a drug substance or drug product.

As used herein, a “batch” of a protein preparation (e.g., a glycoprotein preparation) refers to a single manufacturing run of a protein (e.g., a glycoprotein). Evaluation of different batches thus means evaluation of different manufacturing runs.

As used herein, a “sample” refers to a portion or aliquot. Samples can be separately procured, for example, from different batches or at different time points from the same batch. In some embodiments, evaluation of separate samples may include evaluation of different commercially available containers or vials of the same batch or from different batches.

As used herein, “acquire” or “acquiring” (e.g., “acquiring information”) means obtaining possession of a physical entity, or a value, e.g., a numerical value, by “directly acquiring” or “indirectly acquiring” a physical entity or value. “Directly acquiring” means performing a process (e.g., performing an assay or test on a sample) to obtain a physical entity or value. “Indirectly acquiring” refers to receiving a physical entity or value from another party or source (e.g., a third party laboratory that directly acquired a physical entity or value). “Directly acquiring” a value includes performing a process that includes a physical change in a sample or another substance, e.g., performing an analytical process (e.g., an MS process) which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of an analyte; or by changing the structure of a reagent, or a fragment or other derivative thereof, e.g., by breaking or forming a covalent or non-covalent bond, between a first and a second atom of a reagent.

As used herein, the term “approximately” or “about,” as applied to one or more values of interest, refers to a value that is similar to a stated reference value. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of a stated reference value.

As used herein, the terms “biologic,” “biotherapeutic,” and “biologic product” can be used interchangeably to refer to peptide and protein products. For example, biologics herein include naturally derived or recombinant products expressed in cells, such as, e.g., proteins, glycoproteins, fusion proteins, growth factors, vaccines, blood factors, thrombolytic agents, hormones, interferons, interleukin based products, monospecific (e.g., monoclonal) antibodies, and therapeutic enzymes. Some biologics may be approved under a “Biologics License Application” or “BLA,” under section 351(a) of the Public Health Service (PHS) Act, whereas biosimilar and interchangeable biologics referencing a BLA as a reference product are licensed under section 351(k) of the PHS Act. Section 351 of the PHS Act is codified as 42 U.S.C. 262. Other biologics may be approved under section 505(b)(1) of the Federal Food and Cosmetic Act, or as abbreviated applications under sections 505(b)(2) and 505(j) of the Hatch Waxman Act, where section 505 is codified 21 U.S.C. 355.

As used herein, “approval” refers to a procedure by which a regulatory entity, e.g., the FDA or EMEA, approves a candidate for therapeutic or diagnostic use in humans or animals. As used herein, a “primary approval process” is an approval process that does not refer to a previously approved protein, e.g., it does not require that a protein being approved have structural or functional similarity to a previously approved protein, e.g., a previously approved protein having the same primary amino acid sequence or a primary amino acid sequence that differs by no more than 1, 2, 3, 4, 5, or 10 residues or that has 98% or more sequence identity. In some embodiments, a primary approval process is one in which an applicant does not rely, for approval, on data, e.g., clinical data, from a previously approved product. Exemplary primary approval processes can include, in the U.S., a Biologics License Application (BLA), or supplemental Biologics License Application (sBLA), a New Drug Application (NDA) under 505(b)(1) of the Federal Food and Cosmetic Act, and in Europe an approval in accordance with the provisions of Article 8(3) of the European Directive 2001/83/EC, or an analogous proceeding in other countries or jurisdictions.

As used herein, a “secondary approval process” is an approval process that refers to clinical data for a previously approved product. In some embodiments, a secondary approval can require that a product being approved have structural and/or functional similarity to a previously approved product, e.g., a previously approved protein having the same primary amino acid sequence or a primary amino acid sequence that differs by no more than 1, 2, 3, 4, 5, or 10 amino acid residues or that has at least 98%, 99% or more (100%) sequence identity. In some embodiments, a secondary approval process may be one in which an applicant relies, for approval, on clinical data from a previously approved product. Exemplary secondary approval processes can include, in the U.S., an approval under 351(k) of the Public Health Service Act or under section 505(j) or 505(b)(2) of the Hatch Waxman Act and in Europe, an application in accordance with the provisions of Article 10, e.g., Article 10(4), of the European Directive 2001/83/EC, or an analogous proceeding in other countries or jurisdictions.

As used herein, a “target protein” is any protein of interest to which comparison with a second or “test” protein is desired. An exemplary target protein is an antibody, e.g., a CDR-grafted, humanized or human antibody. Other target proteins can include glycoproteins, cytokines, hematopoietic proteins, soluble receptor fragments, growth factors, and glycoprotein conjugates (e.g., Fc fusion proteins). In some embodiments, a target protein may be a commercially available, or approved, biologic that defines or provides a basis against which a test protein is measured or evaluated. In some embodiments, a target protein may be commercially available for therapeutic use in humans or animals. In some embodiments, a target protein may be approved for use in humans or animals by a primary approval process. In some embodiments, a target protein may be a reference listed drug for a secondary approval process. Exemplary target proteins include those described herein.

An “MS signal,” as used herein, refers to a signal or representation obtained from mass spectrometry (“MS”) and associated with presence of a chemical compound and/or one or more structural characteristics and/or peptides. In some embodiments, an MS signal is a peak, or point therein, in an MS spectrum or chromatogram.

An “oxonium ion,” as used herein, refers to a compound (e.g., a glycopeptide, glycan, or monosaccharide) including an oxygen cation having three bonds.

An “oxonium ion profile,” as used herein, refers to one or more MS signals associated with one or more oxonium ion-containing fragments. In some embodiments, an MS signal in an oxonium ion profile may be expressed as a relative abundance at a retention time.

The term “corresponding oxonium ion profile,” as used herein, refers to two or more oxonium ion profiles that each includes one or more MS signals in the same value range, e.g., a retention time range or mass-to-charge ratio range. In some embodiments, two or more oxonium ion profiles that each includes MS signals for a mass-to-charge ratio range of about 204.08-204.1 are corresponding oxonium ion profiles. In some embodiments, two or more oxonium ion profiles that each includes an MS signal at or about 274.09 are corresponding oxonium ion profiles.

“Tolerable,” as used herein, refers to a range of acceptability for a difference between two or more oxonium ion profiles, e.g., an oxonium ion profile of test protein and a corresponding oxonium ion profile of a target protein. In some instances, a comparison herein is an assessment or measure of variability between two or more oxonium ion profiles, and such compared oxonium ion profiles are tolerable if the variability between them does not exceed (e.g., as determined using a given statistical method) the variability of oxonium ion profiles determined for multiple distinct batches (e.g., 2, 3, 4, 5, or more batches) of such target protein. In some instances, a comparison is tolerable if it meets (e.g., is less than) a predetermined value (e.g., obtained by assessing multiple batches of target protein, as described above). In some instances, comparison of oxonium ion profiles is performed using a representation.

The term “corresponding peptides,” as used herein, refers to two or more peptides having the same amino acid sequence. In some embodiments, corresponding peptides refer to peptides from different samples of the same protein (e.g., a test protein or a target protein) having the same amino acid sequence. In some embodiments, corresponding peptides refer to peptides from a test protein and a target protein having the same amino acid sequence. For example, a peptide from a test protein and a peptide from a target protein are corresponding peptides if they have the same amino acid sequence.

The term “Hex,” as used herein, refers to a hexose, which can be an aldohexose or a ketohexose. Examples of a Hex include, but are not limited to, an allose, an altrose, a glucose, a mannose, a gulose, a idose, a galactose, a talose, a psicose, a fructose, a sorbose, or a tagatose.

The term “HexNAc,” as used herein, refers to an N-acetylhexosamine. A HexNAc can be, for example, a GlcNAc (N-acetylglucosamine) or a GalNAc (N-acetylgalactosamine).

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls. The section headings used herein are for organizational purposes only and are not to be construed as limiting the subject matter described in any way.

These, and other aspects of the disclosed inventions, are described in more detail below and in the claims.

### BRIEF DESCRIPTION OF THE DRAWINGS

FIG. 1 is an experimental workflow of a standard LC-MS/MS methodology.

FIG. 2 is an experimental workflow of a method as described herein. The spectrum at the bottom of the experimental workflow shows an extracted HexNAc oxonium ion profile.

FIG. 3 includes a mass spectrum with a HexNAc oxonium ion structure and a sialic acid oxonium ion structure shown above the respective peaks in the mass spectrum.

FIG. 4 includes panel A: an overlaid extracted HexNAc oxonium ion chromatograms, and panel B: an overlaid extracted sialic acid oxonium ion chromatograms with loess smoothing and parametric time warping, both of which were produced by samples of a model Fc fusion protein from four different CHO cell line clones expressing a model Fc fusion protein (Sample 1, Sample 2, Sample 3 and Sample 4) and on reference samples from three commercial lots of the model Fc fusion protein (RP1, RP2, and RP3). All chromatograms were normalized to the maximum ion intensity.

FIG. 5 is a zoomed-in view of an extracted HexNAc oxonium ion chromatogram (m/z 204.08-204.10) produced by samples of a model Fc fusion protein from four different CHO cell line clones expressing a model Fc fusion protein (Sample 1, Sample 2, Sample 3 and Sample 4) and on reference samples from three commercial lots of the model Fc fusion protein (RP1, RP2, and RP3).

FIG. 6 represents an assessment of glycopeptide oxonium ion dissimilarity performed by calculating differences between binned regions of a chromatogram. FIG. 6, panel A includes overlaid extracted HexNAc oxonium ion chromatograms (m/z 204.08-204.10) produced by samples of a model Fc fusion protein from four different CHO cell line clones expressing a model Fc fusion protein (Sample 1, Sample 2, Sample 3 and Sample 4) and on reference samples from three commercial lots of the model Fc fusion protein (RP1, RP2, and RP3), where the overlaid chromatograms have been binned by minute. FIG. 6, panel B is a heat map indicating overall dissimilarity between the samples and reference samples as calculated from binned sections of the overlaid extracted HexNAc oxonium ion chromatograms in FIG. 6A. FIG. 6, panel C is a heat map indicating overall dissimilarity between the samples and reference samples as calculated from binned sections of the overlaid extracted sialic acid oxonium ion chromatograms (not shown). Samples with the most similarity are purple colored (darker), and samples with the least similarity are peach colored (lighter).

FIG. 7 is a bar graph showing traditional relative quantitation of major glycopeptide species at a specific amino acid site for samples of a model Fc fusion protein from three different CHO cell line clones expressing an model Fc fusion protein (Sample 1, Sample 2, and Sample 3) and on reference samples from two commercial lots of the model Fc fusion protein (RP1 and RP2). Percent abundances were calculated from the extracted ion MS1 area for signal associated with a known glycopeptide.

FIG. 8 includes chromatograms used for the rapid identification of Fab glycosylation in mAbs by HexNAc oxonium ion profiling. Panel A includes a chromatogram showing that mAb1 only has Fc glycosylation. Panel B includes a chromatogram showing that mAb2 has both Fc and Fab glycosylation. These data demonstrate that oxonium ion profiling can be utilized to assess the presence or absence of site-specific glycosylation.

FIG. 9 includes a comparison of a base peak chromatogram (panel A), a HexNAc oxonium ion profile (panel B), and a sialic acid oxonium ion profile (panel C) of an Fc fusion reference sample. The square and diamond represent HexNAc and sialic acid oxonium ions, respectively.

FIG. 10 includes site-specific HexNAc oxonium ion chromatograms of Glycopeptide Site 1 for a reference sample (panel A), Sample 2 (panel B), and Sample 4 (panel C). Glycan nomenclature was as follows: HexNAc(#)Hex(#)Fuc(#)NeuAc(#)NeuGc(#).

FIG. 11 includes an automated similarity assessment of an Fc fusion protein reference sample and samples. The similarity/differences assessments were calculated from processed HexNAc oxonium ion chromatograms (panel A), conventional analysis of all known and detected glycopeptides (panel B), processed sialic acid oxonium ion chromatograms (panel C), and conventional analysis of all known and detected sialic acid-containing glycopeptides (panel D). In the heat maps, reference samples/samples with the most similarity are darker in color, and samples with the least similarity are lighter in color.

FIG. 12 includes bar graphs representing conventional analysis of a priori identified glycopeptide species for each glycosylation site on an Fc fusion reference protein. Percent abundances were calculated from the extracted ion MS1 areas of known glycopeptides.

FIG. 13 includes site-specific HexNAc oxonium ion chromatograms of Glycopeptide Site 4 for an Fc fusion reference sample (panel A), Sample 4 (panel B) and Sample 2 (panel C). Glycan nomenclature was as follows: HexNAc(#)Hex(#)Fuc(#)NeuAc(#)NeuGc(#).

FIG. 14 includes raw traces of HexNAc (panel A) and sialic acid (panel B) oxonium ion chromatograms normalized to the highest peak. Vertical dashes indicate time ranges corresponding to each glycosylation site.

FIG. 15 includes a dot plot showing the selection of parameters for loess smoothing. Raw crossvalidation error of loess regression for linear or quadratic fit and range of span values. The results were from 3 independent trials of 10-fold crossvalidation. Crossvalidation error was noticeably variable across samples as well as between HexNAc and sialic acid oxonium profiles. Span (a) values of 0:002-0:003 and 0:003-0:005 corresponded to the lowest cross-validation errors for the linear and quadratic fits, respectively.

FIG. 16 includes a dot plot showing the selection of parameters for loess smoothing. Crossvalidation error of loess regression from FIG. 15 scaled to the mean of zero and standard deviation of one for each sample and m/z range. Second degree polynomials and the span of 0.004 approximately minimize the error across all samples and m/z ranges.

FIG. 17 includes chromatograms showing examples of parametric time warping effect on HexNAc oxonium ion profiles. Average of RP1, RP2 and RP3 was used as the reference profile.

FIG. 18 includes chromatograms showing examples of parametric time warping effect on sialic acid oxonium ion profiles. Average of RP1, RP2 and RP3 was used as the reference profile.

FIG. 19 includes heat maps showing the assessment of site-specific glycopeptide oxonium ion dissimilarity of Fc fusion protein references samples and samples by calculating differences between HexNAc (left column) and sialic acid (right column) oxonium ion chromatograms. Reference samples/samples with the most similarity are darker in color, and samples with the least similarity are lighter in color.

FIG. 20 includes HexNAc oxonium ion profiles for each glycosylation site, which have been loess smoothed and adjusted for retention time differences between runs using parametric time warping.

FIG. 21 includes sialic acid oxonium ion profiles for each glycosylation site, which have been loess smoothed and adjusted for retention time differences between runs using parametric time warping.

### DETAILED DESCRIPTION

Protein glycosylation plays an important role in a variety of cellular functions, and many protein-based biotherapeutics contain sites along the protein backbone where heterogeneous glycan moieties reside. (Moremen, K. W., et al., Nature Reviews Molecular Cell Biology (2012) 13, 448-462; Spiro, R. G., Glycobiology (2002) 12, 43R-56R). For example, modulation of effector functions via Fc glycosylation has been shown to affect target cell killing mechanisms such as antibody-dependent cellular cytotoxicity (ADCC) and complement-dependent cytotoxicity (CDC). (Jefferis, R., Nature Reviews Drug Discovery (2009) 8, 226-234; Natsume, A., et al., Drug Des Devel Ther (2009) 3, 7-16; Kellner, C., et al., Methods (2014) 65, 105-113). Because glycosylation is a determinant of the function and efficacy for therapeutic proteins, characterization of glycoforms and glycosylation sites of therapeutic proteins is an important part of drug development.

Monoclonal antibodies are the most commonly prescribed biotherapeutic, and usually contain only one N-glycosylation site on the Fc-domain of the protein. For analyzing such antibodies, reducing end modification of enzymatically released N-glycans, followed by high performance liquid chromatography (HPLC) remains a popular technique for glycan characterization. Many important classes of biologics, however, contain multiple sites of both N- and O-linked glycosylation. (Zhu, L., et al. (2014) Taylor & Francis, 1474-1485; Houel, S., et al., Anal. Chem. (2013) 86, 576-584; Larsen, C. P., et al., American Journal of Transplantation (2005) 5, 443-453; Balaratnasingam, C., et al., Clinical ophthalmology (Auckland, NZ) (2015) 9, 2355). For example, many of the marketed Fc-fusion proteins contain five or more glycosylation sites. (Zhu (2014); Houel (2013); Larsen (2005); Balaratnasingam (2015); Berry, J. D., Therapeutic Fc-Fusion Proteins, 217-232). The emergence of antibody and antibody-like molecules with multiple glycan attachment sites has rendered glycan analysis increasingly more complicated with the presence of each additional glycosylation site.

Analyzing multiple glycan attachment sites using established glycopeptide methodologies presents challenges. For instance, by solely characterizing these complex molecules by glycan release-based methods, all glycoforms from all sites become pooled together, thus all information on glycosylation site-specificity is lost. Furthermore, 0-glycans still prove to be difficult to remove from the protein by both enzymatic and chemical procedures. (Takahashi, K., et al., Molecular & Cellular Proteomics (2010) 9, 2545-2557; Wada, Y., et al., Molecular & Cellular Proteomics (2010) 9, 719-727; Christiansen, M. N., et al., Anal. Chem. (2010) 82, 3500-3509).

In addition, established glycopeptide methodologies have generally utilized a priori knowledge of the glycosylation states of the investigated protein(s), database searching of results generated from data-dependent liquid chromatography-tandem mass spectrometry workflows, and extracted ion quantitation of the individual identified species. The inherent complexity of glycosylation, however, makes predicting all glycoforms on all glycosylation sites extremely challenging. As a result, such analyses are typically limited to known glycans.

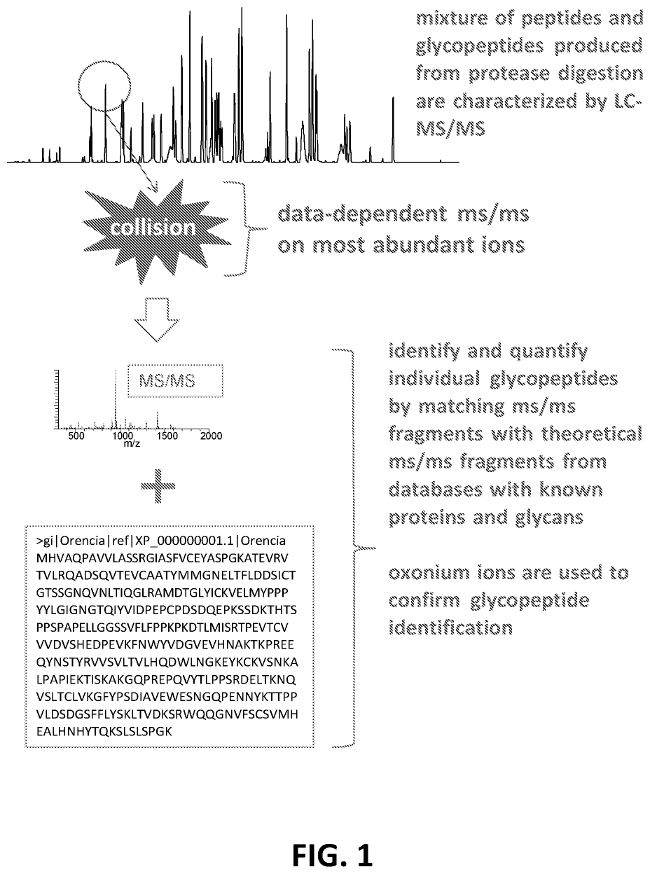

A common methodology for assessing site-specific glycosylation is by liquid chromatography-tandem mass spectrometry (LC-MS/MS). Typically, unknown non-glycosylated peptides and glycopeptides are fragmented separately by various MS/MS techniques (see, e.g., FIG. 1). The fragmentation patterns (MS/MS spectra) can then be searched against a database containing peptide and glycan sequences, in an attempt to identify individual glycopeptides. In some such methods, the presence of low-mass, glycan-specific fragment ions (e.g., oxonium ions) may not be initially analyzed or identified, for example, because the methodology is often data-dependent and analyzes the most abundant ions. Instead, low-mass, glycan-specific fragment ions have traditionally been used after an initial identification of glycopeptides to confirm the initial identification.

While this experimental workflow can work well in certain applications, complete characterization of all glycoforms and glycosylation sites can be challenging because it was very difficult to predict all glycosylated species, and insufficient fragmentation patterns of glycopeptides hinders direct identification from MS/MS spectra. A substantial number of false positives and true negatives have been observed due to, e.g., the fact that not all glycopeptide species are contained in reference databases and poor fragmentation patterns may be generated, even when more advanced MS/MS techniques, such as Electron Transfer Dissociation (ETD) and ultraviolet photodissociation (UVPD), are employed. (Desaire, H. Molecular & Cellular Proteomics (2013) 12, 893-901; Mechref, Y. Current protocols in protein science (2012) 12.11. 11-12.11. 11; Saba, J., et al., International journal of proteomics (2012); Singh, C., et al., J. Proteome Res. (2012) 11, 4517-4525; Zhang, L. and Reilly, J. P., J. Proteome Res. (2008) 8, 734-742; Madsen, J. A., et al., Anal. Chem. (2013) 85, 9253-9261). Moreover, because conventional MS methodologies often use data-dependent peak-picking, MS is only performed on the most abundant ions. While there are advantages to using such methodologies, these methodologies may not detect less abundant, but still important, ions (e.g., ions associated with unknown or unique glycopeptides). Because of these shortcomings, it can be difficult to assign unambiguously all potential sites of glycosylation and their associated glycans for complicated glycoproteins. In turn, it can be difficult, using standard methods, to compare the glycosylation of some glycoproteins (e.g., test proteins) to the glycosylation of another glycoprotein (e.g., a reference protein, e.g., a target protein).

Data-independent analysis of glycopeptides has been applied to glycopeptide analysis to circumvent the traditional reliance on data-dependent acquisition of LC-MS/MS data, which relies on peak picking of the most abundant species from the MS1 spectra to initiate MS/MS, a process that can yield incomplete site-specific characterization. (Sanda, M. and Goldman, R. Anal. Chem. (2016) 88, 10118-10125; Sanda, M., et al., Anal. Bioanal. Chem. (2017) 409, 619-627; Pan, K.-T., et al., Anal. Chem. (2017) 89, 4532-4539). Data-independent analysis techniques fragment all precursor ions in a specified m/z range resulting in a potentially more complete and reproducible data procurement. Quantitation of the glycopeptides (identified by any MS means) can be generally performed by ion extraction of individual glycosylated species from the MS1 and/or MS/MS data. Targeting of low-mass, glycan-specific oxonium ions generated by various MS/MS techniques can be an especially useful technique for deciphering glycopeptides from non-glycosylated species. These ions have been utilized in numerous advantageous approaches to qualitatively and quantitatively assess site-specific glycosylation. (Saba, J., et al., International journal of proteomics (2012); Singh, C., et al., J. Proteome Res. (2012)11, 4517-4525; Cao, L., et al., Anal. Biochem. (2014) 452, 96-102; Toyama, A., et al., Anal. Chem. (2012) 84, 9655-9662; Song, E., et al., Rapid Commun. Mass Spectrom. (2012) 26, 1941-1954; Hart-Smith, G. and Raftery, M. J., J. Am. Soc. Mass Spectrom. (2012) 23, 124-140; Halim, A., et al., J. Proteome Res. (2014) 13, 6024-6032; Nasir, W., et al., J. Proteome Res. (2016) 15, 2826-2840). Nonetheless, even with the advancements made in glycoprotein analysis, it remains difficult to unambiguously assign most, if not all, potential sites of glycosylation and their associated glycans for complicated multiglycosylated proteins. Therefore, currently the known glycoforms and glycosylation sites are assessed during biotherapeutic characterization, which can be a potentially problematic analytical strategy, especially when developing drugs such as biosimilars.

The present disclosure is based, in part, on the discovery that comparisons of glycosylation profiles (e.g., site-specific glycosylation profiles) between glycoproteins can be improved in some circumstances by comparing oxonium ion profiles of the glycoproteins, e.g., when fragmentation of the glycoproteins generates glycopeptides that are difficult to predict and/or identify. The present disclosure describes that MS can be used to generate an oxonium ion profile for a protein (e.g., a glycoprotein). In some embodiments, an oxonium ion profile of a protein can be used to assess biosimilarity, e.g., to manufacture biosimilar proteins (e.g., a protein approved for use in humans by a secondary approval process). For example, in some embodiments, an oxonium ion profile of a sample (e.g., of a test protein) can be compared to an oxonium ion profile of a reference sample (e.g., of a target protein) to, e.g., assess whether the similarity/dissimilarity between the sample and the reference sample is tolerable.

### Overview of Oxonium Ion Profiling

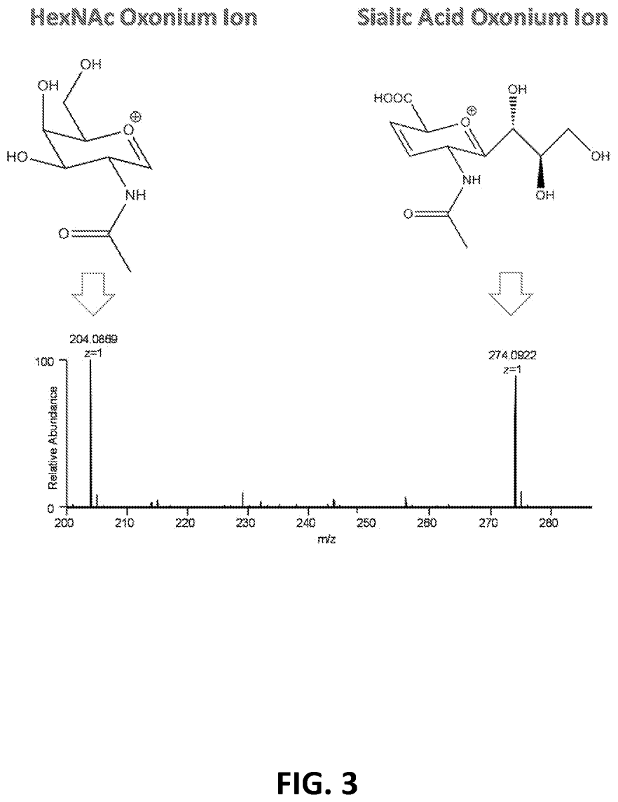

Higher-energy collisional dissociation (HCD) of glycopeptides generates several diagnostic low-mass oxonium ions that can be used for both identification and quantification of glycosylated species. Typical oxonium ions observed include: HexNAc internal fragment (m/z 138), Hex (m/z 163), HexNAc (m/z 204), sialic acid—H₂O (m/z 274), sialic acid (m/z 292), Hex+HexNAc (m/z 366), (among others). The HexNAc (m/z 204) ion is universal to glycopeptides, and can be produced at high abundance under elevated HCD conditions. The sialic acid—H₂O (m/z 274) ion can also be produced with high abundance, and can represent a particularly important acidic sugar that has been shown to alter the anti-inflammatory properties of therapeutic proteins. (Czajkowsky, D. M., et al., EMBO molecular medicine (2012) 4, 1015-1028; Washburn, N., et al., Proceedings of the National Academy of Sciences (2015) 112, E1297-E1306). Therefore, these two ion species can be employed to create the oxonium ion profiles used for biotherapeutic comparisons herein. The structure of the HexNAc and sialic acid ions, and an example mass spectrum of each, can be seen in FIG. 3.

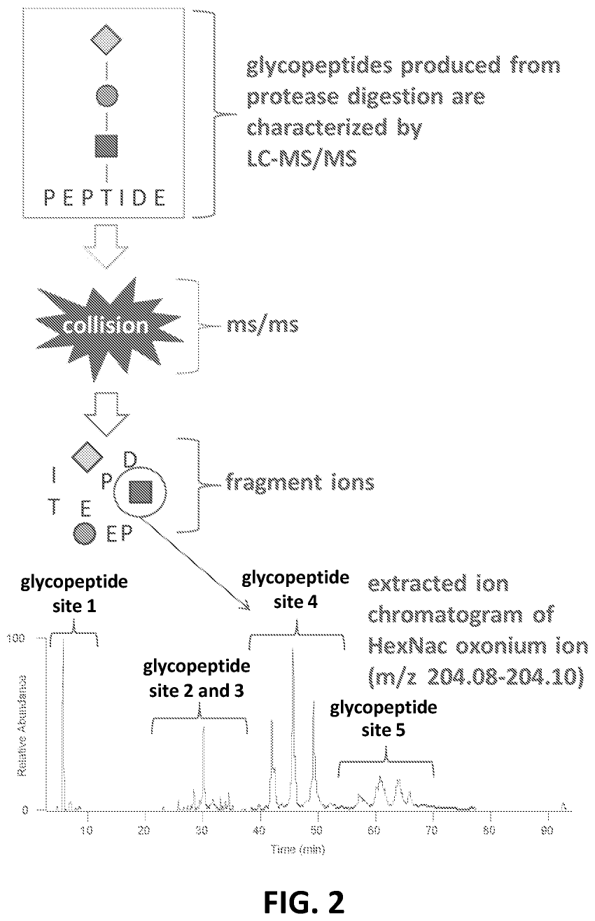

The general methods workflow for oxonium ion profiling can be seen in FIG. 2. In some embodiments, glycosylated samples are first digested, e.g., with trypsin, into a mixture of glycosylated and unglycosylated peptides, which are subsequently analyzed by LC-MS/MS. It is worth noting that other enzymes such as chymotrypsin, AspN, etc. can also be employed for generating glycopeptides, depending on the desired level of experimental rigor. However, trypsin alone is often adequate for producing peptides with acceptable sizes at all glycosylation sites. In some embodiments, reversed-phase chromatography is then utilized for separating the various peptide species. The specific glycosylation locations can largely be distinguished since hydrophobic interactions of the peptide moiety with the stationary phase is expected to dominate the glycopeptide separations. The mass spectrometer can be set to accommodate both all data independent ion fragmentation, as well as conventional data dependent scan events, into a single LC-MS/MS run for optimal sample characterization efficiency; the time contribution of one data independent analysis scan event per cycle of a typical “top-N” data dependent analysis experiment can be quite low. To produce the oxonium ion profiles, the glycan-specific oxonium ions can be extracted across the entirety of the chromatographic separation from the resulting data independent analysis mass spectra.

The conceivable benefits of this methodology include, but are not limited to: (1) most or all precursor ions are fragmented collectively by data independent analysis; therefore, peak picking inconsistencies and high ion abundance bias issues are minimized or eliminated, (2) a priori assumptions about the presence of specific glycosylated species are not required, which better ensures that unknown glycopeptides contribute signal to the overall measurement of similarity, and (3) profile similarity can be quickly and directly assessed between samples (e.g., a reference protein compared to a biosimilar, etc.). That is, while it may be impossible to unambiguously predict and/or identify all glycopeptides in a given sample, the unknown glycosylated species can at least be specifically and reproducibly quantified between samples.

### Analysis Methods

Methods described herein utilize mass spectrometry (MS), a general technique known in the art. Mass spectrometry obtains molecular weight and structural information on chemical compounds by ionizing the molecules and measuring either their time-of-flight or a response of the molecular trajectories to electric and/or magnetic fields. The methods of the present disclosure employ conventional mass spectrometry techniques known to those of skill in the art, and any known MS method can be adapted for use in methods of the disclosure. Exemplary MS can include, but are not limited to, tandem MS (MS/MS), LC-MS, LC-MS/MS, matrix assisted laser desorption ionisation mass spectrometry (MALDI-MS), Fourier transform mass spectrometry (FTMS), ion mobility separation with mass spectrometry (IMS-MS), electron transfer dissociation (ETD-MS), and combinations thereof. Such methods are described in, e.g., Pitt, Clin. Biochem. Rev. 30:19-34 (2009). Mass spectrometers are known in the art and are commercially available from, e.g., Agilent Inc., Bruker Corporation, Waters, AB Sciex, Shimadzu, and Thermo Scientific.

MS can be used to generate an oxonium ion profile, which can include one or more MS signals associated with one or more oxonium ion-containing fragments. The one or more MS signals can represent values (e.g., retention time, relative abundance of the ion, and/or mass-to-charge ratio) obtained by MS for the one or more oxonium containing fragments. An oxonium ion profile can be generated, for example, by creating a chromatogram showing the relative abundance versus retention time as acquired by MS for oxonium ions having a particular mass-to-charge ratio. The mass-to-charge ratio of exemplary oxonium ions observed is provided in Table 1 (below).

TABLE 1

| Oxonium Ion | m/z value |
|---|---|
| HexNAc internal fragment | 138.06 |
| Hex | 163.06 |
| HexNAc | 204.09 |
| Sialic acid | 274.09 |
| sialic acid + H₂O | 292.10 |
| Hex + HexNAc | 366.14 |

Those skilled in the art will recognize that the measured mass-to-charge ratio for an oxonium ion may vary from the above value based on, e.g., the tolerance of the mass spectrometer used and the isotopic distribution. As such, in some circumstances, it may be advantageous to examine MS signals over a range of mass-to-charge ratios. For example, an oxonium ion profile may include MS signals for oxonium ion-containing fragments that include oxonium ions within the following m/z ranges: (i) m/z 138.05-138.07; (ii) m/z 163.05-163.07; (iii) m/z 204.08-204.10; (iv) m/z 274.08-274.10; (v) m/z 292.09-292.11; (vi) m/z 366.13-366.15; or (vii) combinations thereof.

In some embodiments, a conventional MS spectra is not generated in methods described herein. For example, a plot of the relative abundance of an ion versus the mass-to-charge ratio of the ion is not generated, and therefore, an MS spectra is not used to identify and/or quantify individual glycopeptides. In certain circumstances, an MS spectrum is generated in parallel to the methods disclosed herein.

In some embodiments, higher-energy collisional dissociation (HCD) of glycopeptides can be used to generate several diagnostic low-mass oxonium ions that can be used for both identification and quantification of glycosylated species. The typical ions observed include those ions shown in Table 1 above. The HexNAc (m/z 204) ion is universal to glycopeptides and can be produced at high abundance under elevated HCD conditions. The sialic acid—H₂O (m/z 274) ion can also be generated from a large number glycopeptides, and represents a particularly important acidic sugar that has been shown to alter the anti-inflammatory properties of therapeutic proteins. (Czajkowsky, D. M., et al., EMBO molecular medicine (2012), 4, 1015-1028; Washburn, N., et al., Proceedings of the National Academy of Sciences (2015) 112, E1297-E1306). These two ion species were employed to create the oxonium ion profiles used for biotherapeutic comparisons herein. The structure of the HexNAc and sialic acid ions, and an example mass spectrum of each, can be seen in FIG. 3.

In some embodiments, data independent MS methodologies are used to acquire oxonium ion profiles. Such methods can increase the likelihood that most, if not all, glycopeptide species are accounted for in an analysis. For example, data independent methodologies (e.g., all ion fragmentation) can be advantageous because such methodologies are less dependent on a priori assumptions about what glycopeptides are present in a sample. Such methodologies can also facilitate comparisons of a sample (e.g., of a test protein) to a reference sample (e.g., of a target protein), e.g., where the sample and/or the reference sample includes an unknown or unique glycopeptide.

In some methods of the disclosure, a glycosylated protein (e.g., a test protein, e.g., a biotherapeutic) is digested (e.g., using a protease, e.g., trypsin, chymotrypsin, AspN) into a mixture of peptides and glycopeptides (see, e.g., FIG. 2). The mixture of peptides and glycopeptides is then analyzed by LC-MS/MS. Before peptides and glycopeptides are introduced into an MS instrument, they are subjected to data-independent all ion fragmentation to break apart the peptide species emerging from a liquid chromatography column. Data obtained from glycan oxonium ions are then extracted with high mass accuracy from MS signals generated during the performance of MS to produce an analytical response that is specific to glycan-modified peptides. In some instances, oxonium ion profiles of a first protein (e.g., a test protein) and a second protein (e.g., a target protein) can be compared to assess similarity/dissimilarity of the first protein to the second protein, e.g., during one or more stages of process development and/or production of a biosimilar product including the first protein. In one exemplary method, MS is used to assess the similarity of a test biologic to a reference biologic that is approved under a BLA.

In some instances, higher-order structure of a protein can be assessed by performing MS on a protein (e.g., a sample of a protein preparation, more specifically, a sample of a glycoprotein preparation) to obtain a mass spectrum of relative abundance of ions with a particular mass-to-charge ratio over a given range (e.g., 100 to 2000 amu). Numerous methods for determining amount/abundance of a peptide from an amount/abundance of an ion are known to those of ordinary skill in the art. For example, relative abundance of a given ion may be compared to a table that converts that relative abundance to an absolute amount of a peptide. Additionally or alternatively, external standards may be run with samples, and a standard curve constructed based on ions generated from such standards. Using a standard curve, relative abundance of a given ion may be converted into an absolute amount of a peptide. Methods of generating and using such standard curves are well known in the art, and one of ordinary skill is capable of selecting an appropriate internal standard.

### Applications

In some instances, methods disclosed herein can be used to confirm the identity and/or quality of a protein, e.g., glycoprotein preparation. For example, methods can include assessing preparations (e.g., samples, lots, and/or batches) of a test protein, e.g., to confirm whether a test protein qualifies as a target protein, and, optionally, qualifying a test protein as a target protein if qualifying criteria (e.g., predefined qualifying criteria) are met; thereby evaluating, identifying, and/or producing (e.g., manufacturing) a protein product.

Methods of the disclosure can have a variety of applications and can include, e.g., quality control at different stages of manufacture, analysis of a protein preparation prior to and/or after completion of manufacture (e.g., prior to or after distribution to a fill/finish environment or facility), and/or prior to and/or after release into commerce (e.g., before distribution to a pharmacy, a caregiver, a patient, or other end-user). In some instances, a protein preparation may be a drug substance (i.e., an active pharmaceutical ingredient or “API”) or a drug product (i.e., an API formulated for use in a subject such as a human patient). In some instances, a protein preparation may be from a stage of manufacture or use that is prior to release to care givers or other end-users; prior to packaging into individual dosage forms, such as syringes, pens, vials, or multi-dose vials; prior to determination that a batch can be commercially released, prior to production of a Certificate of Testing, Material Safety Data Sheet (MSDS) or Certificate of Analysis (CofA) of a preparation. In some instances, a protein preparation may be from an intermediate step in production, e.g., it is after secretion of a protein from a cell but prior to purification of drug substance.

Evaluations from methods described herein can be useful for guiding, controlling or implementing one or more of a number of activities or steps in a process of making, distributing, and monitoring and providing for a safe and efficacious use of a protein preparation. Thus, in some embodiments, e.g., responsive to an evaluation, e.g., depending on whether a criterion is met, a decision or step is taken. Methods can further include one or both of a decision to take a step and/or carrying out the step itself. For example, a step can include one in which a preparation (or another preparation for which the preparation is representative) is: classified; selected; accepted or discarded; released or processed into a drug product; rendered unusable for commercial release, e.g., by labeling it, sequestering it, or destroying it; passed on to a subsequent step in manufacture; reprocessed (e.g., a preparation may undergo a repetition of a previous process step or subjected to a corrective process); formulated, e.g., into drug substance or drug product; combined with another component, e.g., an excipient, buffer or diluent; disposed into a container; divided into smaller aliquots, e.g., unit doses, or multi-dose containers; combined with another preparation of a protein (e.g., the same protein); packaged; shipped; moved to a different location; combined with another element to form a kit; combined, e.g., placed into a package with a delivery device, diluent, or package insert; released into commerce; sold or offered for sale; delivered to a care giver or other end-user; or administered to a subject. For example, based on a result of a determination or whether one or more subject entities is present, or upon comparison to a reference standard, a batch from which a preparation is taken can be processed, e.g., as just described.

Methods described herein may include making a decision: (a) as to whether a protein preparation may be formulated into drug substance or drug product; (b) as to whether a protein preparation may be reprocessed (e.g., a preparation may undergo a repetition of a previous process step); and/or (c) that a protein preparation may not be suitable for formulation into drug substance or drug product. In some instances, methods can include: formulating as referred to in step (a), reprocessing as referred to in step (b), or rendering a preparation unusable for commercial release, e.g., by labeling it or destroying it, as referred to in step (c).

### Test Proteins and Target Proteins

Methods described herein can be used to make and/or evaluate a test protein preparation, e.g., a test biologic preparation. In some embodiments, a test protein can be a test biologic being evaluated for similarity to a target protein, e.g., a target biologic. A test biologic may or may not be commercially available. In some embodiments, a test biologic is not commercially available for therapeutic use in humans or animals. In some embodiments, a test biologic has not been approved for therapeutic or diagnostic use in humans or animals. In some embodiments, a test biologic has been approved, e.g., under a secondary approval process, for therapeutic or diagnostic use in humans or animals. In some embodiments, a test protein (e.g., test biologic) has the same primary amino acid sequence as a target protein (e.g., target biologic) or differs by no more than 1, 2, 3, 4, 5, 10, 15, 20, 25, 30 residues and/or has at least 90, 95, 98, 99% or is identical to a target protein sequence (e.g., target biologic sequence). The terms the “same primary amino acid sequence,” “a primary amino acid sequence that differs by no more than 1, 2, 3, 4, 5, 10, 15, 20, 25, or 30 residues,” “sequences that have at least 98% or more sequence identity,” or similar terms, relate to level of identity between a primary amino acid sequence, e.g., of first protein, e.g., a test protein, and a primary amino acid sequence, e.g., of second protein, e.g., a target protein. In some embodiments, a protein preparation or product includes amino acid variants, e.g., species that differ at terminal residues, e.g., at one or two terminal residues. In some embodiments of such cases, a sequence identity comparison is between a primary amino acid sequence of the most abundant (e.g., the most abundant active) species in each of the products being compared. In some embodiments, sequence identity refers to an amino acid sequence encoded by a nucleic acid that can be used to make the product.

Nonlimiting, exemplary target proteins can include abatacept (Orencia®, Bristol-Myers Squibb), abciximab (ReoPro®, Roche), adalimumab (Humira®, Bristol-Myers Squibb), aflibercept (Eylea®, Regeneron Pharmaceuticals), alefacept (Amevive®, Astellas Pharma), alemtuzumab (Campath®, Genzyme/Bayer), basiliximab (Simulect®, Novartis), belatacept (Nulojix®, Bristol-Myers Squibb), belimumab (Benlysta®, GlaxoSmithKline), bevacizumab (Avastin®, Roche), canakinumab (Ilaris®, Novartis), brentuximab vedotin (Adcetris®, Seattle Genetics), certolizumab (CIMZIA®, UCB, Brussels, Belgium), cetuximab (Erbitux®, Merck-Serono), daclizumab (Zenapax®, Hoffmann-La Roche), denileukin diftitox (Ontak®, Eisai), denosumab (Prolia®, Amgen; Xgeva®, Amgen), eculizumab (Soliris®, Alexion Pharmaceuticals), efalizumab (Raptiva®, Genentech), etanercept (Enbrel®, Amgen-Pfizer), gemtuzumab (Mylotarg®, Pfizer), golimumab (Simponi®, Janssen), ibritumomab (Zevalin®, Spectrum Pharmaceuticals), infliximab (Remicade®, Centocor), ipilimumab (Yervoy™, Bristol-Myers Squibb), muromonab (Orthoclone OKT3®, Janssen-Cilag), natalizumab (Tysabri®, Biogen Idec, Elan), ofatumumab (Arzerra®, GlaxoSmithKline), omalizumab (Xolair®, Novartis), palivizumab (Synagis®, MedImmune), panitumumab (Vectibix®, Amgen), ranibizumab (Lucentis®, Genentech), rilonacept (Arcalyst®, Regeneron Pharmaceuticals), rituximab (MabThera®, Roche), tocilizumab (Actemra®, Genentech; RoActemra, Hoffman-La Roche) tositumomab (Bexxar®, GlaxoSmithKline), trastuzumab (Herceptin®, Roche), and ustekinumab (Stelara®, Janssen).

Antibodies

In some instances, test proteins and target proteins described herein are antibodies. As used herein, the term “antibody” refers to a polypeptide that includes at least one immunoglobulin variable region domain, e.g., an amino acid sequence for an immunoglobulin variable region domain or immunoglobulin variable region domain sequence. For example, an antibody can include a heavy (H) chain variable region domain (abbreviated herein as V_{H}), and a light (L) chain variable region domain (abbreviated herein as V_{L}). In another example, an antibody can include two heavy chain variable region domains and two light chain variable region domains. The term “antibody” encompasses antigen-binding fragments of antibodies (e.g., single chain antibodies, Fab, F(ab′)₂, Fd, Fv, and dAb fragments) as well as complete antibodies, e.g., intact immunoglobulins of types IgA, IgG, IgE, IgD, IgM (as well as subtypes thereof). Light chains, light chain variable regions or fragments thereof can be of types kappa or lambda. In some embodiments, an antibody includes an Fc region. In some embodiments, an antibody is a therapeutic antibody.

Antibodies described herein can include, for example, monoclonal antibodies, polyclonal antibodies (e.g., IVIG), multi-specific antibodies (e.g., bispecific antibodies), human antibodies, humanized antibodies, camelized antibodies, chimeric antibodies, single-chain Fvs (scFv), disulfide-linked Fvs (sdFv), and anti-idiotypic (anti-Id) antibodies, and antigen-binding fragments of any of the above. Antibodies can be of any type (e.g., IgG, IgE, IgM, IgD, IgA and IgY), class (e.g., IgG1, IgG2, IgG3, IgG4, IgA1 and IgA2) or subclass.

Antibodies or fragments thereof can be produced by any method known in the art for synthesizing antibodies (see, e.g., Harlow, et al., Antibodies: A Laboratory Manual, (Cold Spring Harbor Laboratory Press, 2nd ed. 1988); Brinkman, et al., 1995, J. Immunol. Methods 182:41-50; WO 92/22324; WO 98/46645). Chimeric antibodies can be produced using methods described in, e.g., Morrison, 1985, Science 229:1202, and humanized antibodies by methods described in, e.g., U.S. Pat. No. 6,180,370.

Glycoprotein Conjugates

In some instances, test proteins and target proteins can be glycoprotein conjugates (e.g., Fc regions or Fc fragments containing one or more N-glycosylation sites thereof that are conjugated or fused to one or more heterologous moieties). Heterologous moieties can include, but are not limited to, peptides, polypeptides, proteins, fusion proteins, nucleic acid molecules, small molecules, mimetic agents, synthetic drugs, inorganic molecules, and organic molecules. In some instances, a glycoprotein conjugate can be a fusion protein that includes a peptide, polypeptide, protein scaffold, scFv, dsFv, diabody, Tandab, or an antibody mimetic fused to an Fc region, such as a glycosylated Fc region. A fusion protein can include a linker region connecting an Fc region to a heterologous moiety (see, e.g., Hallewell, et al. (1989), J. Biol. Chem. 264, 5260-5268; Alfthan, et al. (1995), Protein Eng. 8, 725-731; Robinson & Sauer (1996)).

### Recombinant Gene Expression

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994)).

In some embodiments, a protein described herein can be produced using recombinant methods. Recombinant expression of a gene, such as a gene encoding a polypeptide, such as an antibody described herein, can include construction of an expression vector containing a polynucleotide that encodes a polypeptide. Once a polynucleotide has been obtained, a vector for the production of a polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems can include microorganisms, such as bacteria (e.g., E. coli and B. subtilis), transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g. Ti plasmid) containing polypeptide coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., an adenovirus late promoter; a vaccinia virus 7.5K promoter).

For bacterial systems, a number of expression vectors can be used, including, but not limited to, an E. coli expression vector pUR278 (Ruther, et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). Efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes a gene product in a specific fashion desired. Different host cells may have characteristic and specific mechanisms for post-translational processing and modification of proteins and gene products. Appropriate cell lines or host systems can be chosen to ensure the correct modification and processing of a polypeptide expressed. Such cells can include, for example, established mammalian cell lines and insect cell lines, animal cells, fungal cells, and yeast cells. Mammalian host cells can include, but are not limited to, CHO, VERY, BHK, HeLa, COS, MDCK, 293, 3T3, W138, BT483, Hs578T, HTB2, BT20 and T47D, NSO (a murine myeloma cell line that does not endogenously produce any immunoglobulin chains), CRL7O3O and HsS78Bst cells.

For long-term, high-yield production of recombinant proteins, host cells can be engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a protein described herein been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultrafiltration, salting-out and dialysis procedures (see Antibodies: A Laboratory Manual, Ed Harlow, David Lane, Cold Spring Harbor Laboratory, 1988). Further, as described herein, a glycoprotein can be fused to heterologous polypeptide sequences to facilitate purification. Glycoproteins having desired sugar chains can be separated with a lectin column by methods known in the art (see, e.g., WO 02/30954).

### Pharmaceutical Compositions

A protein (e.g., an antibody) described herein can be incorporated into a pharmaceutical composition. Such a pharmaceutical composition can be useful in prevention and/or treatment of diseases. Pharmaceutical compositions including a polypeptide (e.g., an antibody) can be formulated by methods known to those skilled in the art (see, e.g., Remington's Pharmaceutical Sciences, 20th Ed., Lippincott Williams & Wilkins, 2000). A pharmaceutical composition can be administered parenterally in the form of an injectable formulation including a sterile solution or suspension in water or another pharmaceutically acceptable liquid. For example, a pharmaceutical composition can be formulated by suitably combining a polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. An amount of active ingredient included in a pharmaceutical preparation can be such that a suitable dose within a designated range is provided.

Route of administration can be, e.g., parenteral, for example, administration by injection, transnasal administration, transpulmonary administration, or transcutaneous administration. Additionally or alternatively, administration can be systemic or local by intravenous injection, intramuscular injection, intraperitoneal injection, subcutaneous injection.

A suitable means of administration can be selected based on factors, such as, the age and condition of a patient. A single dose of a pharmaceutical composition containing a polypeptide (e.g., antibody) can be selected from a range of 0.001 mg/kg of body weight to 1000 mg/kg of body weight. On the other hand, a dose can be selected in the range of 0.001 mg/kg of body weight to 100000 mg/kg of body weight, but the present disclosure is not limited to such ranges. Dosages and methods of administration can vary depending on the weight, age, condition, and the like of a patient, and can be suitably selected as needed by those skilled in the art.

The disclosure is further illustrated by the following examples. The examples are provided for illustrative purposes only. They are not to be construed as limiting the scope or content of the disclosure in any way.

### EXAMPLES

### Example 1: Glycan Oxonium Profiling Method

This Example describes an exemplary method of producing an oxonium ion profile for a glycoprotein using an LC-MS/MS analysis of all digestion fragments from a glycoprotein. In one exemplary method, a preparation of a glycosylated model Fc fusion protein was first subjected to protease digestion to produce a mixture of peptides and glycopeptides (see FIG. 2). The mixture was then analyzed by liquid chromatography-tandem mass spectrometry (LC-MS/MS). As the glycopeptides came off the liquid chromatography column and were introduced into the mass spectrometry instrument, data-independent all ion fragmentation was used to break apart all peptide species. The fragments generated by all ion fragmentation were then analyzed using a mass spectrometer and spectra were recorded.

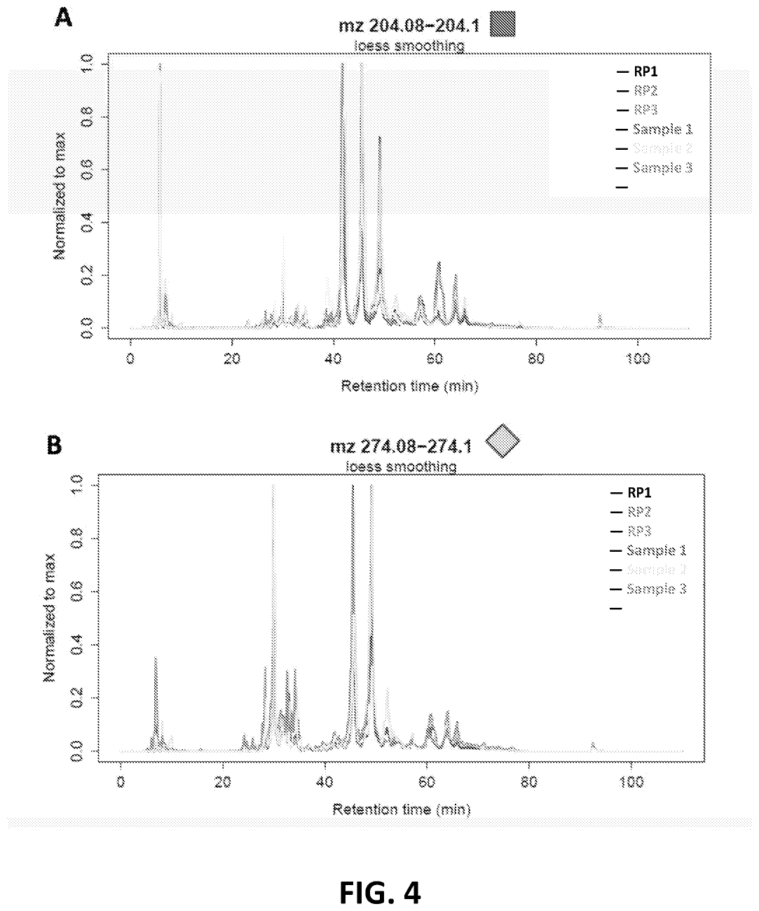

The data corresponding to glycan oxonium ions were then extracted with high mass accuracy from the resulting mass spectra to create chromatograms (“oxonium ion profiles”) that were specific to glycan-modified peptides. FIG. 4, panels A and B show glycan oxonium ion profiles that were generated by extracting data in the range of m/z 204.08-204.1, which encompasses the signal corresponding to [HexNAc−water]+generally found at m/z 204.09, and in the range of m/z 274.08-274.1, which encompasses the signal corresponding to [sialic acid−water]+generally found at m/z 274.09 (see FIG. 3).

In addition, peaks in the oxonium ion profile were assigned to glycosylation sites on the model Fc fusion protein based on the chromatographic retention time. Thus, as shown in FIG. 2, for example, this exemplary method can provide site-specific information about glycans on glycoproteins. Other exemplary techniques for assigning glycosylation sites include electron capture dissociation (ECD), electron transfer dissociation (ETD), and photodissociation.

### Example 2: Glycan Oxonium Ion Profiling of Samples of a Model Fc Fusion Protein

This Example shows that glycan oxonium ions profiles can be used to detect and analyze the similarity/dissimilarity of glycans and glycopeptides obtained from different glycoprotein preparations (e.g., samples or reference samples). Using the methodology described in Example 1, glycan oxonium ion profiling was conducted on samples of a model Fc fusion protein from four different CHO cell line clones expressing the model Fc fusion protein (Sample 1, Sample 2, Sample 3 and Sample 4) and on reference samples from three commercial lots of the model Fc fusion protein (RP1, RP2, and RP3). Exemplary chromatograms are depicted in FIG. 4, panels A and B.

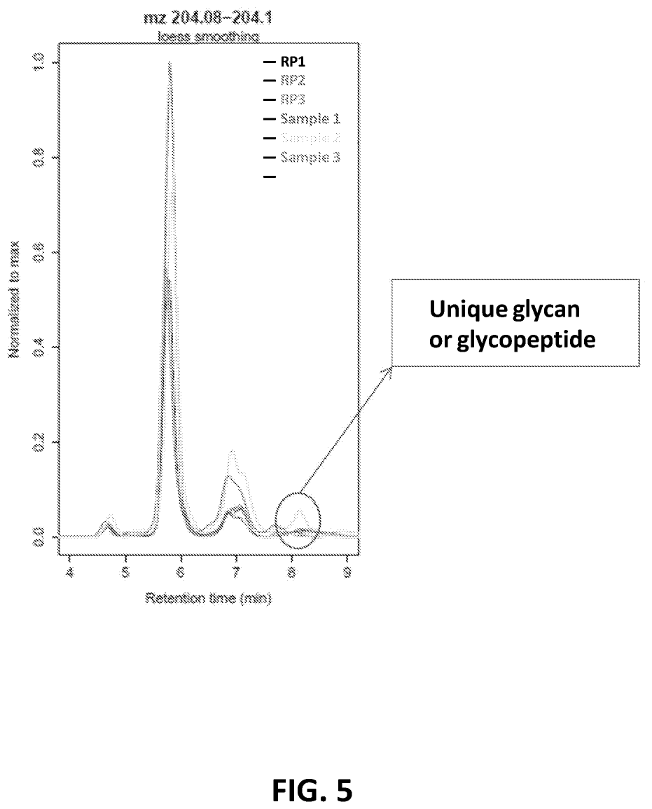

The chromatograms illustrate that glycan oxonium ion profiles, for example as obtained by the method described in Example 1, can be used to discern differences between the glycans and glycopeptides in different glycoprotein preparations. For example, a unique glycan or glycopeptide was detected in Sample 2 that was not detected in any other samples or reference samples. This observation is highlighted in FIG. 5, which is an expanded view of peaks from FIG. 4, panel A. Without being bound to any particular theory, the circled peak may represent glycosylation at an aberrant protein site and/or the presence of an unusual glycan, the detection of which can be used to ascertain differences between this Fc fusion protein and, e.g., a reference sample of the Fc fusion protein. In some embodiments, further analysis of the peak is performed, which may include multiple stages of tandem mass spectrometry, photodissociation, and/or electron-based MS/MS techniques such as electron transfer dissociation (ETD) and electron capture dissociation (ECD).

### Example 3: Quantification of Differences in Glycan Oxonium Ion Profiles for Samples of a Model Fc Fusion Protein

This Example describes a method for quantifying the similarity/dissimilarity of glycans and glycopeptides obtained from different glycoprotein preparations (e.g., samples or reference samples). As demonstrated in Example 2, visual inspection of glycan oxonium ion profiles can be used to detect differences in glycoprotein samples. In some embodiments, statistical measures of similarity/dissimilarity can be used to quantitate the similarity/dissimilarity.

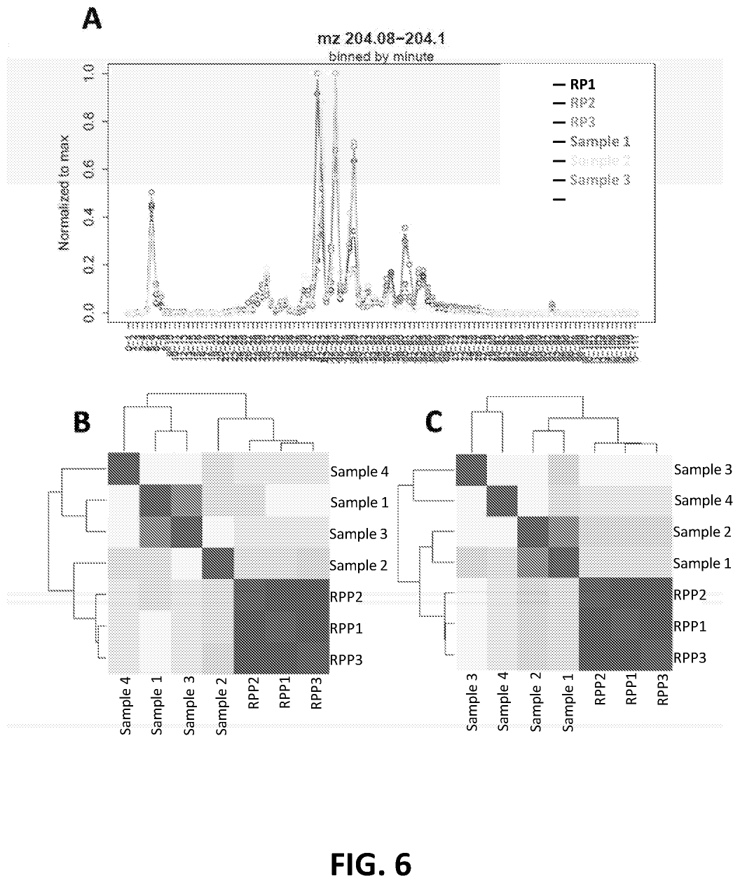

In one exemplary method, an algorithm that separates each ion profile into specified “bins” based on retention time was created. The algorithm was written in the R programming language. As shown in FIG. 6, panel A, the HexNAc oxonium ion chromatograms (m/z 204.08-204.10) of the four Fc fusion protein clone samples and the three samples of the reference Fc fusion protein were binned by minute. These binned regions were assessed for all samples and used to flag sections of similarity/dissimilarity. The overall similarity/dissimilarity of samples was calculated from binned data obtained from the HexNAc and sialic acid oxonium ion chromatograms, as shown in the heat maps of FIG. 6, panels B and C, respectively. In these heat maps, samples that were the most similar are represented by increasingly dark colors, while those that were the most dissimilar are represented by lighter colors. For example, the replicate samples of the reference Fc fusion protein were the most similar to each other, as indicated by the darkest boxes in a 3×3 grid found in the lower right corner of both FIG. 6, panels A and B. The similarity between the samples of the reference protein indicated a high degree of reproducibility between the glycan oxonium ion profiles. Conversely, each Fc fusion protein clone demonstrated varying degrees of similarity to the reference protein. It was also observed that the Fc fusion protein clones that were more similar to the reference protein for the HexNAc profiles were often more dissimilar for the sialic acid profiles.

### Example 4: Comparison of Glycan Oxonium Ion Profiling with Standard Analysis Methods

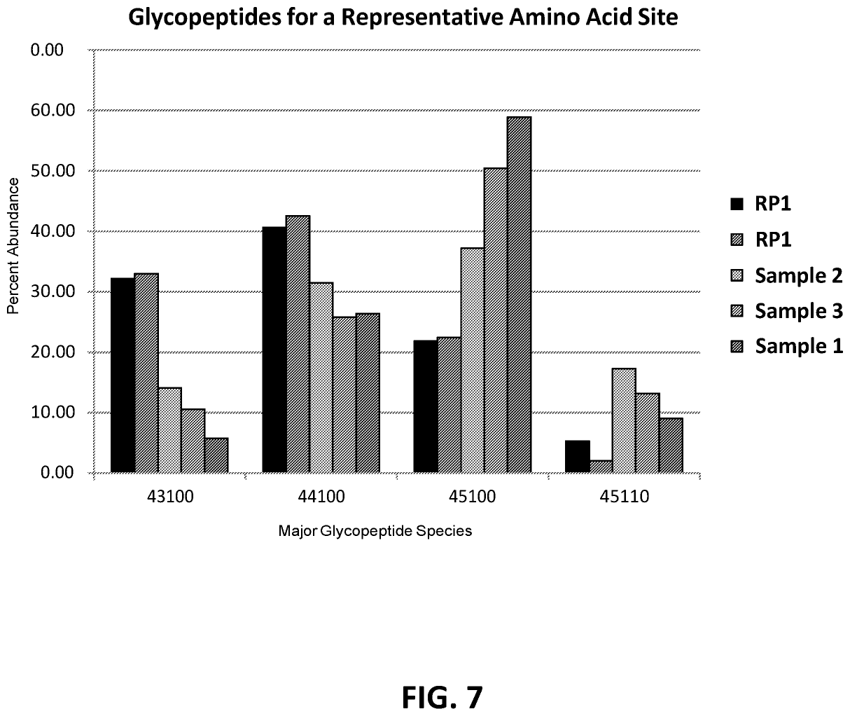

This Example demonstrates that oxonium ion profiling can provide information about the glycans present on a glycoprotein sample (including how those glycans are similar/dissimilar to the glycans on a reference sample) that may be different than the information about the glycans that can be determined by standard LC-MS/MS methodology. Three of the four samples used in Example 2 (Sample 1, Sample 2, and Sample 3) and the three reference samples used in Example 2 (RP1, RP2, and RP3) were analyzed using standard LC-MS/MS for assessing glycosylation (see FIG. 1). First, each sample was protease digested into a mixture of peptides and glycopeptides. The mixture was then subjected to liquid chromatography. Based on the resulting chromatogram, tandem mass spectrometry was conducted on the ions that were most abundant in the mixture. FIG. 7 illustrates the results from the LC-MS/MS. The reported percent abundances were calculated from the extracted ion MS1 areas of the major, known glycopeptides.

The increased resolving power of oxonium ion profiling for assessing similarity/dissimilarity between a sample and a reference sample was demonstrated by comparing the results obtained from oxonium ion profiling to the results obtained from the standard LC-MS/MS method. For example, using the standard LC-MS/MS, Sample 2 had the most similar glycopeptide profile to the reference protein (FIG. 7). However, as discussed in Example 2, oxonium ion profiling of the same sample, Sample 2, identified a peak corresponding to unique glycan or glycopeptide (FIG. 5). The disparity in the results demonstrates that in some instances traditional LC-MS/MS methods may not identify dissimilarity between glycoprotein samples, whereas oxonium ion profiling as described herein can identify such dissimilarities. Accordingly, oxonium ion profiling can be used in place of or to supplement the traditional LC-MS/MS methods.

### Example 5: Glycan Oxonium Ion Profiling Methods

### Sample Preparation

100 μg of a sample or a reference sample was diluted to 1 mg/mL with 6 M guanidine HCL in 20 mM sodium phosphate, 100 mM sodium chloride, pH 7.0, and denatured for 30 minutes at 37° C. Disulfide reduction was then performed by adding 5 mM Dithiothreitol, and incubating for 80 minutes at 37° C. Proteins in the sample or reference sample were then alkylated with 12 mM N-ethylmaleimide for two hours in the dark at room temperature. Using Amicon 10 k spin filters (EMD Millipore, Billerica, Mass.) the sample or reference sample was buffer exchanged into 50 mM ammonium bicarbonate, and digested with 5 μg of trypsin (1:20 enzyme to substrate ratio) for 19 hours at 37° C. Protease reactions were quenched with 2.5% formic acid.

### LC-MS/MS

Tryptic peptides obtained from the samples and/or reference samples (4 μg) were injected onto a 2.1×50 mm (1.7 μm particle size) AQUITY BEH C18 column (Waters, Milford, Mass.) and heated at 50° C. using a Dionex Ultimate 3000 RSLCnano (Santa Clara, Calif.) system. Eluent A and B were 0.1% formic acid in water and 0.1% formic acid in acetonitrile, respectively. Gradient elution was performed at a flow rate of 50 μL/min as follows: linear gradient of 3% eluent B at zero minutes, 7% eluent B at 3 minutes, 13% eluent B at 20 minutes, 28% eluent B at 40 minutes, 35% eluent B at 60 minutes, 50% eluent B at 70 minutes, 80% eluent B from 80 to 90 minutes, and 3% eluent B from 92 to 110 minutes.

The liquid chromatography system was coupled to a Thermo Scientific Q Exactive (Bremen, Germany) for mass spectrometric analysis. The instrument was operated to accommodate both data-independent all ion fragmentation and data-dependent analyses in a single run. Data dependent acquisition was performed as follows: MS1 events were comprised of the positive mass scan at a range of 400-2000 m/z followed by one HCD event at 25% normalize collision energy (NCE) on the most abundant ion from the first event. Dynamic exclusion duration was 20 s with a single repeat count, and charged species ≥7 were excluded. Data-independent all ion fragmentation was performed at a range of 200-2000 m/z, and a NCE of 35%. The electrospray ionization voltage was set to 3.32 kV, capillary temperature was 250° C., sheath gas was 15, and the S-Lens RF level was set to 50 for all analyses. Resolution was 35,000 for MS1 scans, and 17,500 for data dependent acquisition MS/MS and data-independent all ion fragmentation scans. The automated gain control was set to 1E6 with a maximum injection time of 250 ms for both MS1 and data-independent all ion fragmentation, and automated gain control was 2E5 with a maximum injection time of 80 ms for data dependent acquisition MS/MS. An isolation window of 4 m/z was used for data dependent acquisition MS/MS scans. MS1, data-independent all ion fragmentation, and data dependent acquisition MS/MS spectra were produced from 5, 3, and 1 microscans, respectively.

### Data Analysis

HexNAc and sialic acid oxonium ion profiles were generated by extracting out the m/z range of 204.08-204.10 and 274.08-274.10, respectively, across the entire data-independent acquisition for each sample or reference sample. The mass accuracy tolerance for ion extraction was ±0.01 Da for the experiments herein, and proved to be sufficiently specific for the targeted oxonium ions as no obvious interferences were observed.

Conventional targeted glycopeptide analysis was performed by mining LC-MS/MS files of the most abundant known glycoforms and glycosylation sites. Skyline (version 3.5.0.9320) was used to create extracted ion chromatograms (XICs) of each individual species. Results were then reported as a relative abundance percentage for each individual glycosylation site.

Database searching analysis was performed using Byonic, similarly as previously described. (Bern, M., et al., Anal. Chem. (2007) 79, 1393-1400; Bern, M., et al., Curr. Protoc. Bioinformatics (2012) Chapter 13, Unit 1320; Madsen, J. A., et al., Anal. Chem. (2016) 88, 2478-2488). In short, LC-MS/MS files were searched against a protein database composed of the protein of interest. Trypsin was used as the enzyme with up to two allowable missed cleavages. Mass tolerances of 10 ppm and 0.02 Da were used for MS1 and MS/MS spectra, respectively. N-Ethylmaleimide of cysteine was set as a fixed modification for the searches, and glycan databases consisting of 118 N-glycans and 6 O-glycans were used by Byonic for identifying glycopeptides.

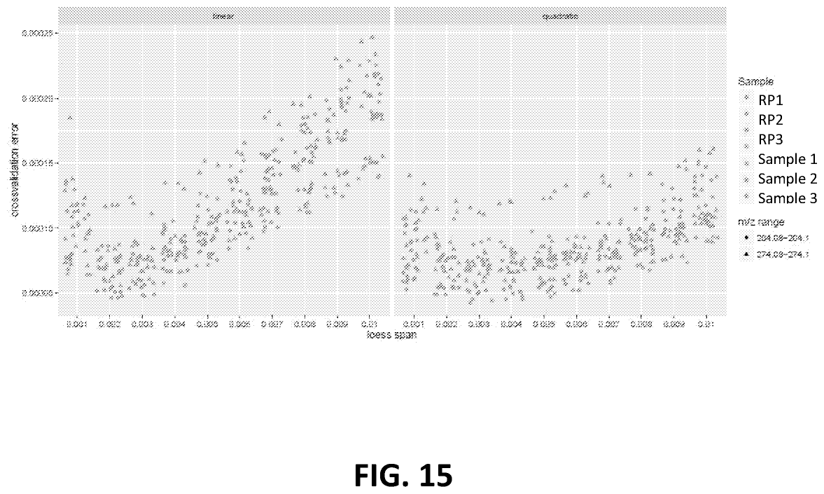

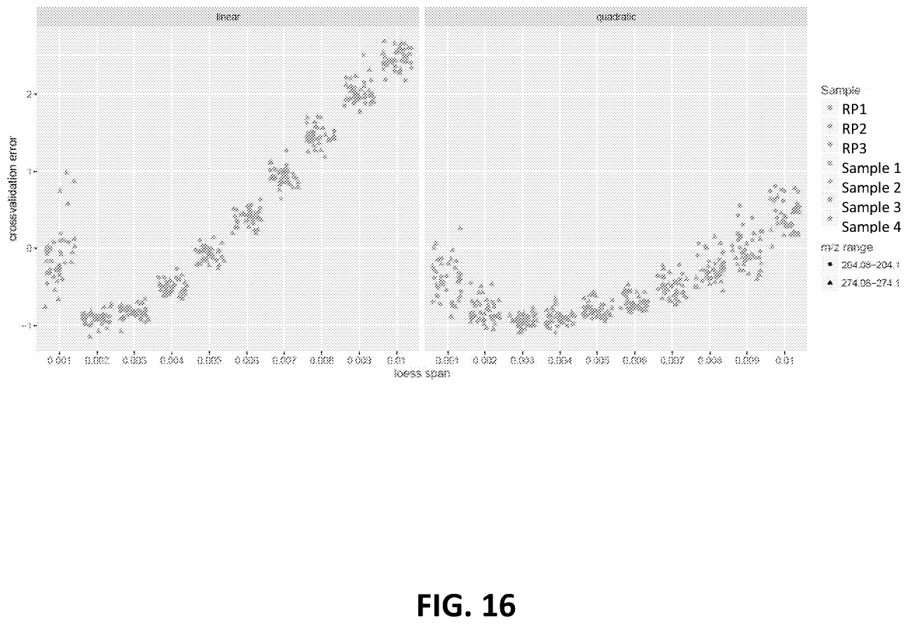

The automated similarity assessment tool was created in the R programming language. (http://www.R-project.org (2014)). Thermo .RAW files were converted to MGF format and loaded into R using MSnbase library (Gatto, L., Lilley, K. S. Bioinformatics (2011) btr645), and extracted ion chromatograms of m/z 204.08-204.10 and 274.08-274.10 were generated from the data-independent scans. The resulting oxonium ion profiles were then loess smoothed (Cleveland, W. S., et al., Statistical models in S (1992) 2, 309-376) and adjusted for differences in retention time by parametric time warping. (Eilers, P. H. Anal. Chem. (2004) 76, 404-411; Bloemberg, T. G., et al., Chemometrics and Intelligent Laboratory Systems (2010) 104, 65-74). Selection of parameters for loess regression was performed by cross-validation as illustrated in FIGS. 15 and 16.

The dissimilarities between each pair of samples and reference samples were calculated as the sum of the absolute differences between resulting oxonium ion relative abundances across the entire time course of the experiment (e.g., from 0-80 minutes) or for time intervals corresponding to selected glycopeptide sites. For the site-specific calculations, the sample dissimilarities were calculated over time intervals of 0-20 minutes (glycopeptide site 1), 20-36 minutes (glycopeptide site 2/3), 36-55 minutes (glycopeptide site 4), and 55-80 minutes (glycopeptide site 5). Heat maps were generated from the 2-way comparison of all Fc-fusion protein samples, and were clustered using average linkage. For direct comparisons to the oxonium ion profiling results, sample dissimilarities from conventional targeted analysis were calculated as sums of absolute differences between relative abundances (normalized to the highest area count) for each individual glycopeptide.

### Session Information

Except for FIGS. 3, 12, and 13, all computational results herein have been obtained in statistical programming environment R. (R Core Team, R: A Language and Environment for Statistical Computing. R Foundation for Statistical Computing, Vienna, Austria, 2014). Numerical and graphical computation results included in this document have been compiled using literate programming capacity supported in R by Sweave (F. Leisch, “Sweave: Dynamic generation of statistical reports using literate data analysis,” in Compstat 2002—Proceedings in Computational Statistics (W. Härdle and B. Rönz, eds.), pp. 575-580, Physica Verlag, Heidelberg, 2002. ISBN 3-7908-1517-9) and further converted into Portable Document Format (PDF) with the LATEXsuite of tools (M. Goossens, et al., The LATEX companion. Addison-Wesley Reading, Massachusetts (1994)). This section contains information about the version of R software and additional packages that have been used throughout this analysis:
R version 3.1.2 (2014-10-31), i386-w64-mingw32
Locale: LC_COLLATE=English_United States.1252, LC CTYPE=English_United States.1252, LC_MONETARY=English_United States.1252, LC_NUMERIC=C, LC_TIME=English_United States.1252
Base packages: base, datasets, graphics, grDevices, methods, parallel, stats, utils
Other packages: Biobase 2.26.0, BiocGenerics 0.12.1, BiocParallel 1.0.0, ggplot2 2.1.0, gplots 2.16.0, MSnbase 1.14.2, mzR 2.0.0, ptw 1.9-11, Rcpp 0.11.3
Loaded via a namespace (and not attached): a_y 1.44.0, a_yio 1.34.0, base64enc 0.1-2, BatchJobs 1.5, BBmisc 1.8, Bioclnstaller 1.16.5, bitops 1.0-6, brew 1.0-6, caTools 1.17.1, checkmate 1.5.1, codetools 0.2-9, colorspace 1.2-4, DBI 0.3.1, digest 0.6.8, doParallel 1.0.10, fail 1.2, foreach 1.4.2, gdata 2.13.3, grid 3.1.2, gtable 0.1.2, gtools 3.4.1, impute 1.40.0, IRanges 2.0.1, iterators 1.0.7, KernSmooth 2.23-13, labeling 0.3, lattice 0.20-29, limma 3.22.1, MALDIquant 1.12, munsell 0.4.2, mzID 1.4.1, nloptr 1.0.4, pcaMethods 1.56.0, plyr 1.8.3, preprocessCore 1.28.0, reshape2 1.4.1, RSQLite 1.0.0, S4Vectors 0.4.0, scales 0.4.0, sendmailR 1.2-1, stats4 3.1.2, stringr 0.6.2, tools 3.1.2, vsn 3.34.0, XML 3.98-1.1, zlibbioc 1.12.0

### Example 6: Rapid Identification of Fab Glycosylation

This Example demonstrates that oxonium ion profiling can be used to quickly detect the presence or absence of glycosylation on multiple glycosylation sites. In particular, this Example demonstrates that oxonium ion profiling can be used to quickly detect the presence or absence of Fab glycosylation, as well as Fc glycosylation.

N-linked Fc glycosylation is well conserved across IgG subclasses; therefore, glycopeptide identification at the Fc region is relatively straightforward using conventional methodologies. Fab glycosylation characterization, conversely, is subject to a number of challenges when conventional methodologies are used. As one example, there are a tremendous number of amino acid combinations that can be present in a Fab variable region; as such, predicting the amino acid sequence would be difficult and predicting the site-specific glycosylation sites would be even harder. As another example, it would be very difficult to predict all the Fab site-specific glycosylation in intravenous immunoglobulin (IVIG) because the IgGs are pooled from thousands of plasma donors. It would also be difficult to foresee how a given expression system might affect Fab glycosylation of recombinantly produced antibodies and/or antibody-like therapeutics. Additionally, O-glycosylation is difficult to predict because a consensus amino acid sequence motif has not been identified. Glycan oxonium profiling can overcome these challenges.

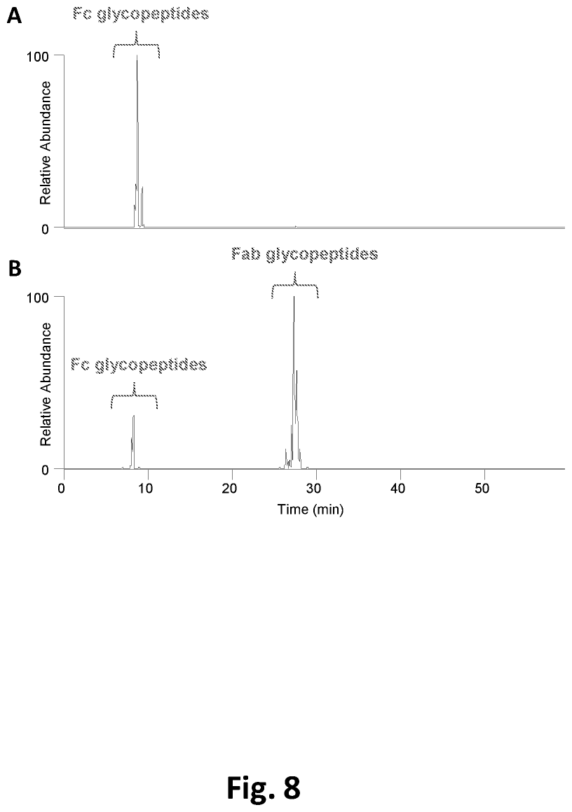

Glycan oxonium profiles were obtained for two samples, mAb1 (which has Fc glycosylation) and mAb2 (which has both Fc and Fb glycosylation), in accordance with the methods described above in Example 5. In this application, the glycan oxonium profiling was utilized to rapidly detect the presence or absence of Fab glycosylation, as shown in FIG. 8, panels A and B. The HexNAc oxonium fingerprint of mAb1 in FIG. 8, panel A, illustrates that only one site of glycosylation is present, which elutes at the same retention time as the conserved Fc N-linked glycopeptides. FIG. 8, panel B, contrastingly, shows two distinct retention time regions with clustered glycopeptide species for mAb2, and highlights the ability of glycan oxonium profiling to quickly screen for less predictable glycosylated species.

A more exhaustive and retention-time targeted database searching procedure was then initiated on the accompanying data dependent analysis spectra revealing that indeed, the detected glycopeptides were from the Fab region (data not shown). In this example, both protein sequences of mAb1 and mAb2 were known; however, for biotherapeutics with less clear Fab sequences and more potential sites of glycosylation, the employment of database searching often becomes significantly less effective.

### Example 7: Comparing a Base Peak Chromatogram to Oxonium Ion Profiles

This Example shows that glycan oxonium ions profiles can be used to determine the number of glycosylation sites present on a glycoprotein in a sample or reference sample. This Example also demonstrates that comparing different glycan oxonium ions profiles (e.g., a HexNAc oxonium ions profile to a sialic acid oxonium ion profile) of a glycoprotein can reveal the relative amounts of the glycans on the glycoprotein and/or at specific glycan sites of the glycoprotein. For this Example, glycan oxonium profiles were obtained for an exemplary Fc fusion protein reference sample in accordance with the methods described above in Example 5.

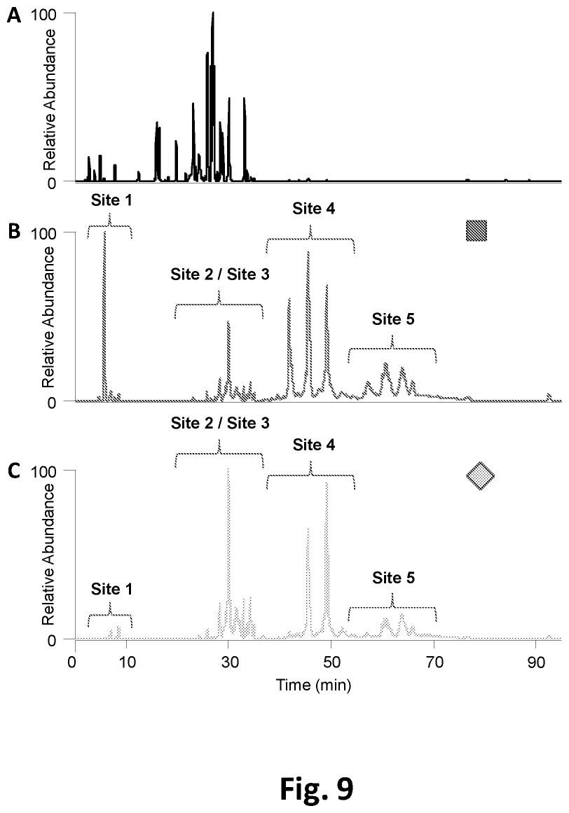

FIG. 9, panels A-C, highlight the differences between the base peak chromatogram, HexNAc oxonium ion profile, and sialic acid oxonium ion profile, respectively, of the reference sample. The base peak chromatogram (MS1) and oxonium fingerprints were extracted from the same trypsin digestion and LC-MS/MS run. The Fc fusion protein has five major glycosylation sites, which indicated that a significant portion of the protein sequence was decorated with glycoforms. The base peak chromatogram (FIG. 9, panel A), however, was still dramatically different than both oxonium ion profiles (FIG. 9, panels B and C). Without being bound by any theory, this result may have arisen because unglycosylated peptides usually ionize better than the glycopeptides. Furthermore, each glycosylation site will be made up of many glycoforms, thus the glycopeptide signal may have been divided amongst the different glycoforms.

The HexNAc and sialic acid oxonium profiles showed four distinct areas where the different glycosylation sites elute (FIG. 9, panels B and C). It is worth noting that glycosylation sites 2 and 3 eluted in the same general region due to their close sequence proximity, which indicated the presence of glycopeptides containing both sites. Obvious differences were observed between the two oxonium profiles. As expected, the sialic acid fingerprint has fewer peaks because not all glycoforms will contain sialic acid. It also had a different signal distribution between sites compared to the HexNAc profile (e.g., site 1 is lower).

Thus, this example demonstrates that oxonium ion profiling can provide site-specific information about glycans on glycoproteins. Moreover, because different oxonium ion profiles can provide useful and different information, multiple oxonium profiles should be analyzed (and, e.g., matched) when assessing samples and/or reference samples.

### Example 8: Identification of Unknown/Unpredicted Glycosylated Species

This Example demonstrates that oxonium ion profiling can provide information about the glycans present on a glycoprotein sample (including how those glycans are similar/dissimilar to the glycans on a reference sample) that may be different than the information about the glycans that can be determined by conventional methodologies (e.g., data dependent LC-MS/MS. This Example also demonstrates that oxonium ion profiling can be used to potentially detect unknown and/or unpredicted glycosylated species. To compare to the oxonium ion profiling methodology to a conventional LC-MS/MS methodology, four different Fc fusion protein clones (Sample 1, Sample 2, Sample 3, and Sample 4), as well as the three replicate reference samples (RP1, RP2, and RP3) were analyzed in accordance with the methods described in Example 5.

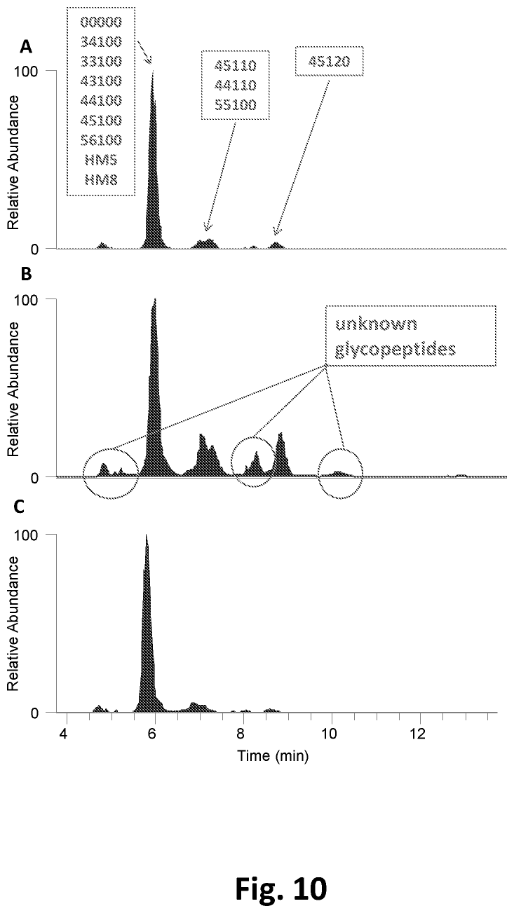

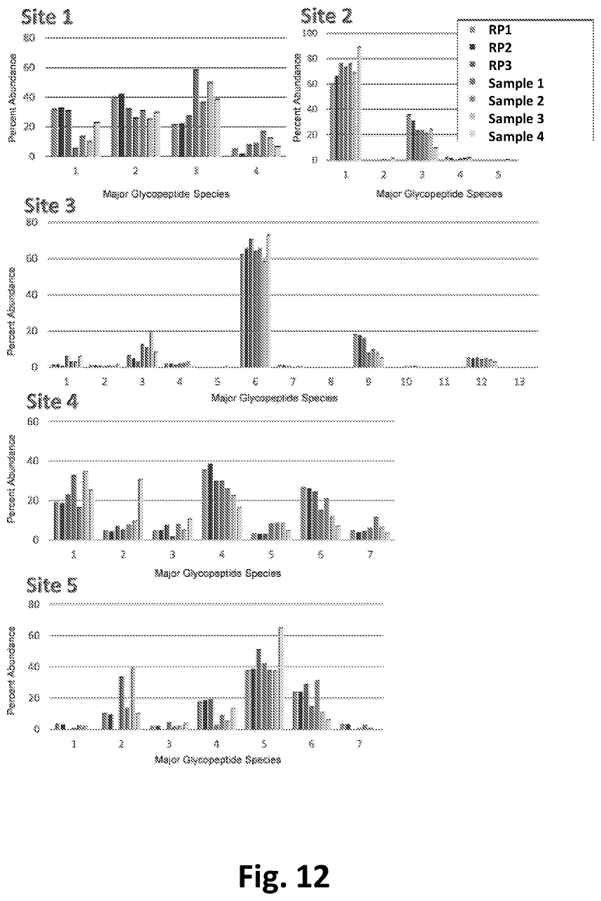

FIG. 12 shows the relative quantitation of the most abundant glycoforms determined to have been present at each glycosylation site. FIG. 10, panels A-C, display the zoomed in, site-specific HexNAc oxonium ion profile comparisons of Site 1 that were obtained for one of the reference sample replicates, Sample 2, and Sample 4. The known glycoforms that were identified are labeled in the top profile.

Turning to the data, unknown glycopeptide species were detected in Sample 2 that were not detected by either conventional analysis or by glycopeptide database searching (data not shown). The unknown glycopeptides could be from glycosylation at an unexpected protein site, unpredictable protein modification, or from a glycopeptide containing an unusual glycan; notably, these species were not observed with sufficient abundance in the reference sample. Conversely, the HexNAc oxonium ion profile of Sample 4 exhibits obvious similarity to the reference sample. In other words, differences were observed between Sample 2 and Sample 4 when glycan oxonium ion profiling was used. This differs from the data obtained from the conventional targeted method (see FIG. 12), which showed that Sample 2 and Sample 4 had relatively similar glycopeptide abundance percentages for Site 1 (see FIG. 12). These data demonstrate that glycan oxonium ion profiling can detect differences in glycoproteins that may not be detected using conventional methodologies.

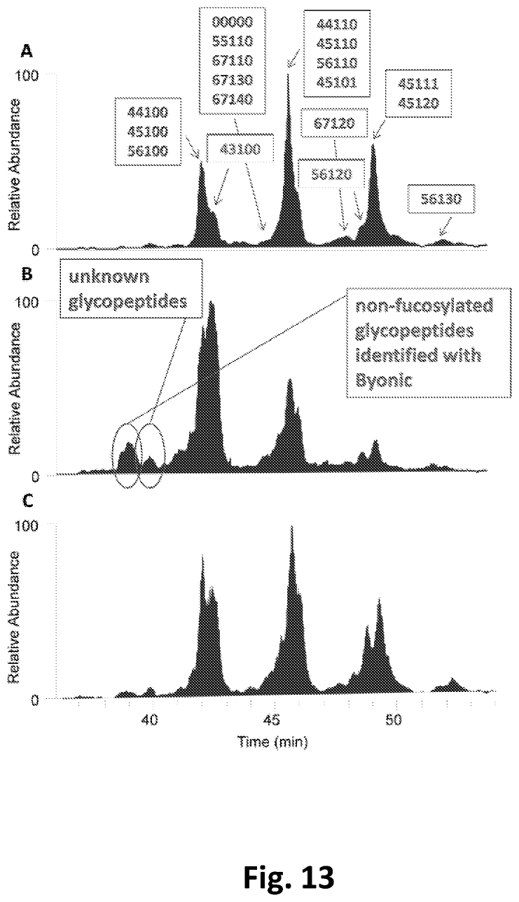

FIG. 13, panels A-C, illustrate another site-specific oxonium ion profile comparison for the reference sample, Sample 2, and Sample 4. In this analysis, site 4 was assessed. Unknown glycopeptide species were again detected, but this time for Sample 4, whereas Sample 2 was highly similar to the reference sample. The conventional targeted method was developed using the reference protein (a typical strategy), and therefore, the non-fucosylated glycopeptides (circled) were not targeted in the sample screening. These species were detected by Byonic database searching, and were observed discernibly in the oxonium ion profile.

In this analysis, Sample 4 had several glycoforms that were substantially different than the reference samples, as well as Sample 2 by conventional targeted analysis (FIG. 12), a result that agrees well with the HexNAc fingerprints. Both site-specific comparisons highlight the power of the oxonium ion profiling technique, and the ability of the method to make meaningful comparisons between samples and/or reference samples. The comparisons also illustrate the challenge of matching all glycosylation sites to a reference protein since certain samples (e.g., clones) will be more similar at some sites, but more dissimilar at others. Therefore, it is critical to have methodology (such as oxonium ion profiling) that can better detect and quantify all potential differences.

### Example 9: Automated Similarity Assessment from Oxonium Ion Profiles

This Example describes a method for quantifying the similarity/dissimilarity of glycans and glycopeptides obtained from different glycoprotein preparations (e.g., samples or reference samples). This Example also shows a method for quantifying the similarity/dissimilarity of glycans and glycopeptides obtained from different glycoprotein preparations (e.g., samples or reference samples) can be performed at a globally or site-specifically. For this Example, four different Fc fusion protein clones (Sample 1, Sample 2, Sample 3, and Sample 4), as well as the three replicate reference samples (RP1, RP2, and RP3) were analyzed in accordance with the methods described in Example 5.

While visually assessing the oxonium profiles may be sufficient for some applications, a rapid and automated assessment of profile similarity between numerous samples will often be warranted. Thus, an algorithm that quantifies the differences of each section of the oxonium profile between samples was developed. All data was directly input into the program, loess smoothed and parametric time warped to eliminate retention time discrepancies between samples. Differences in ion abundances at every second were calculated between a set of profiles.

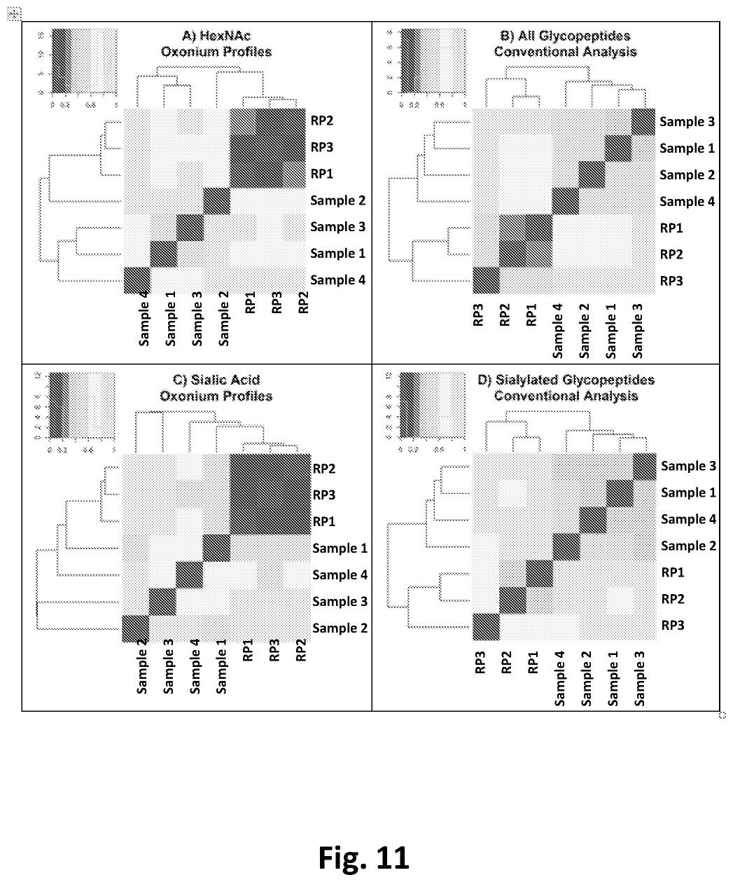

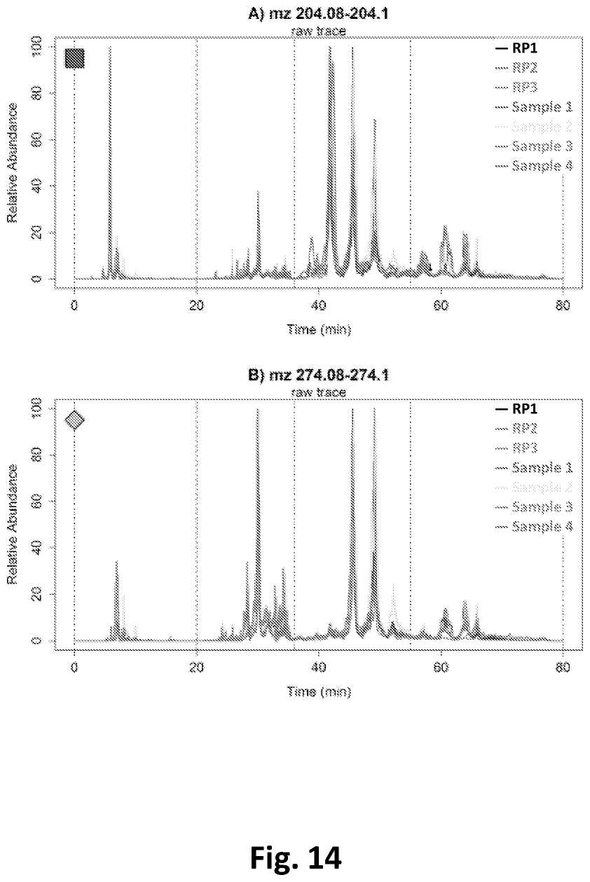

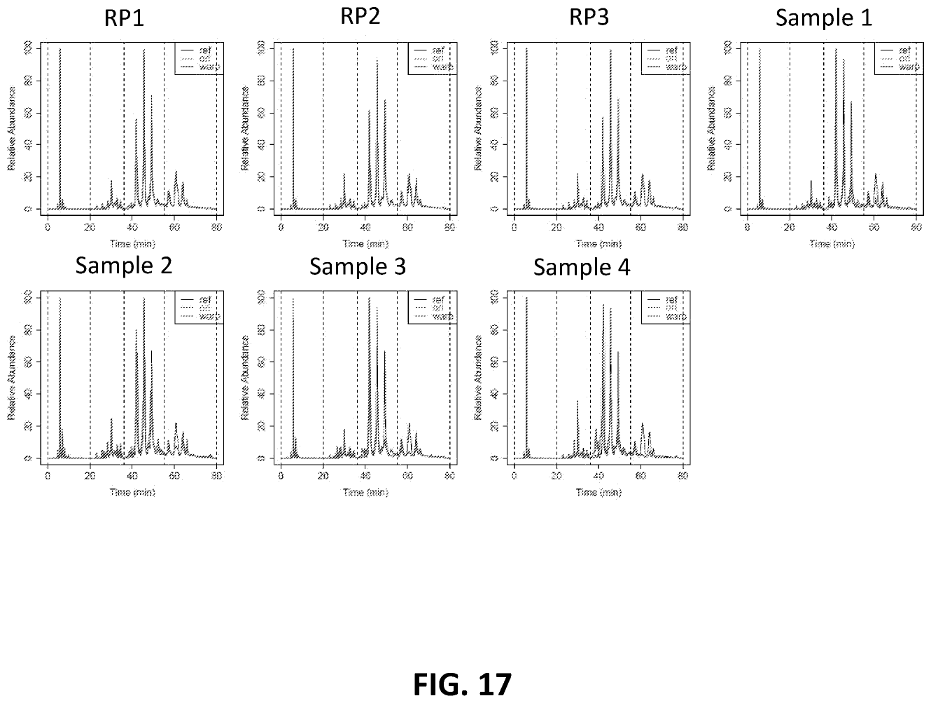

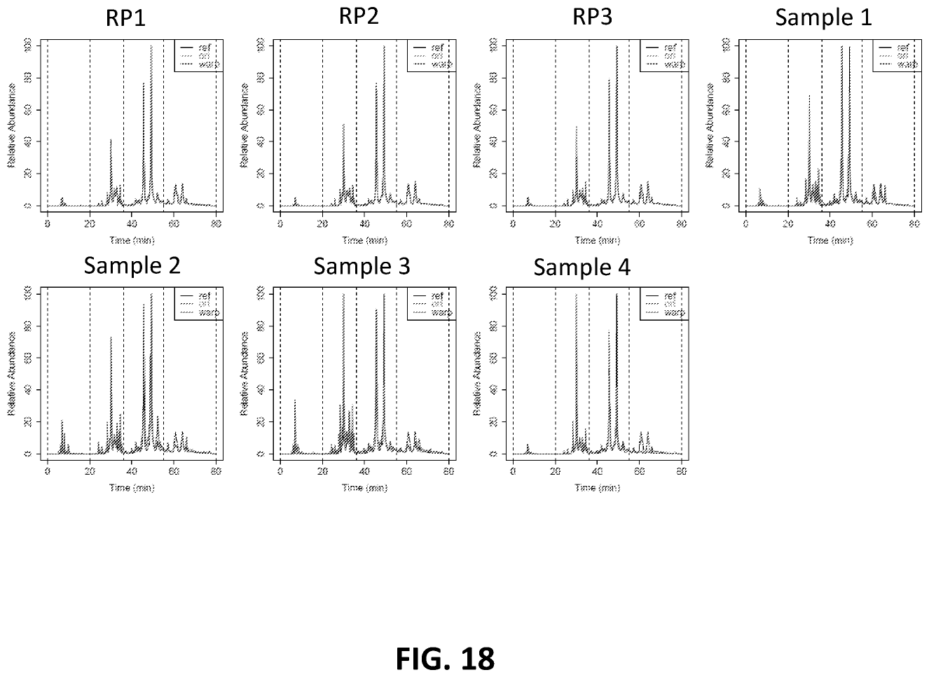

FIG. 4, panels A and B, illustrate the overlaid HexNAc and sialic acid oxonium ion chromatograms of all of the samples and reference samples after loess smoothing and parametric time warping. The corresponding unprocessed profiles can be found in FIG. 14, panels A and B. Selection of the loess smoothing parameters by cross-validation is illustrated in FIGS. 15 and 16, and the parametric time warping effect on HexNAc and sialic acid profiles is illustrated in FIGS. 17 and 18, respectively. The overall glycopeptide similarity assessment is shown in FIG. 11, panels A-D, indicating significant variations in oxonium fingerprints across samples.

To compare between methods, a global difference value was calculated from the conventional targeted results (FIG. 12) by summing all relative abundance differences for each glycoform at every glycosylation site. Only the sialylated species were used to calculate the differences shown in FIG. 11, panel D. For all heat maps, pairs of samples that were the most similar have increasingly darker colors, while those that were the most dissimilar were increasingly lighter colored. Replicates of the references (RP1, RP2, and RP3) were shown to be highly similar for the oxonium ion profiles, illustrating a suitable degree of method reproducibility. Conversely, each sample had varying degrees of resemblance to the reference samples, and interestingly, samples that were more similar to reference samples for the HexNAc profiles were often more dissimilar for the sialic acid profiles (comparing FIG. 11, panels A-C).

The similarity assessment of the conventional targeted data exhibited noticeably less discriminating power between the samples as compared to the oxonium profiles (e.g., conventional analysis yielded data that showed the reference samples were less similar to each other and more similar to the samples). The trends in similarity between the two methods were in agreement for some pair-wise comparisons, and in disagreement for others. For example, Sample 1 and Sample 3 were shown to be highly similar for each method; however, Sample 1 and Sample 4 were dissimilar by HexNAc oxonium profiling and similar by conventional analysis.

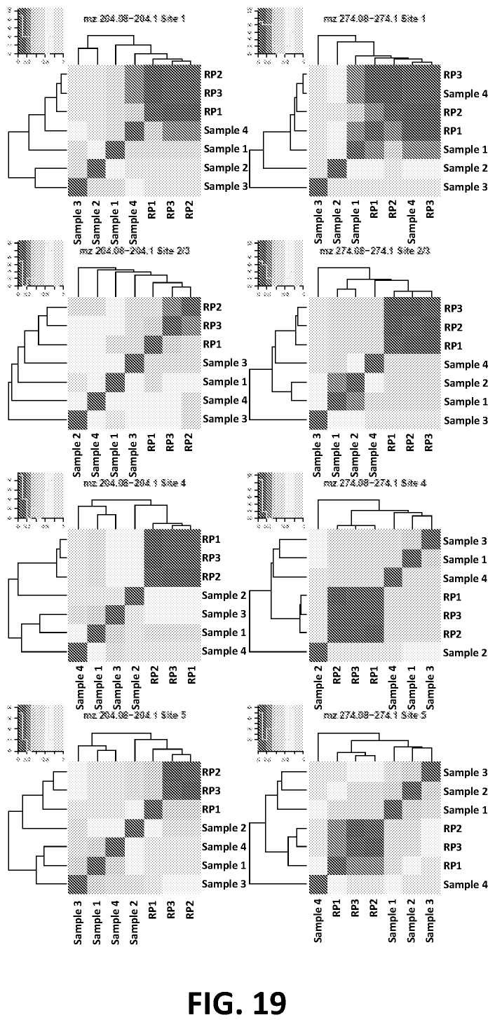

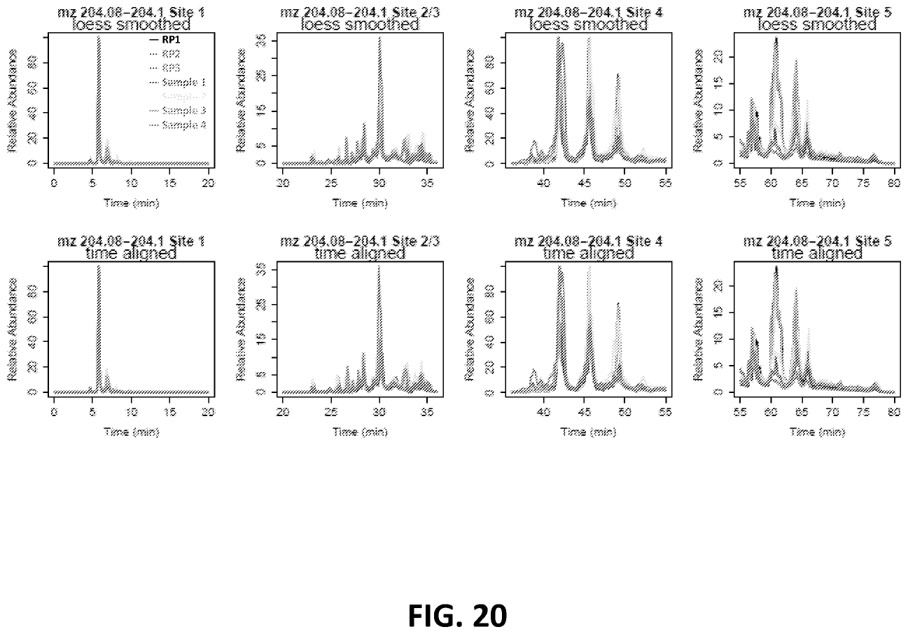

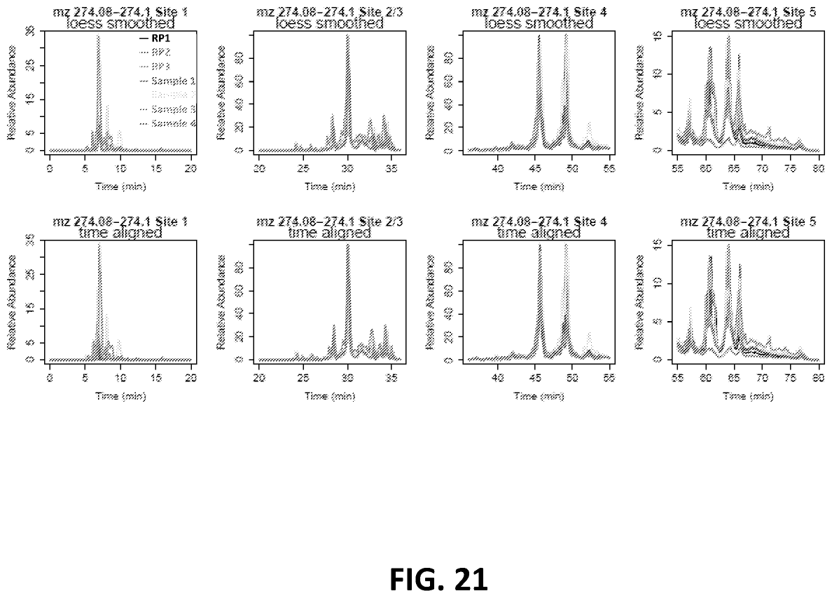

As a final assessment of the oxonium ion profiling method, the automated assessment tool was applied to quantify dissimilarity between samples site-specifically. That is, the HexNAc and sialic acid profiles were divided into sections that represent the various glycosylation sites and the differences between the reference samples and the samples at each site were calculated. The results can be seen in FIG. 19. While significantly more complicated than the global profile comparisons illustrated in FIG. 11, these comparisons provides a higher level of resolution for glycopeptide differentiation. For example, the site-specific automated assessment in FIG. 19 confirms that Sample 4 was more similar to the references as compared to Sample 2 for the HexNAc oxonium ion profiles at Site 1, and that the opposite was true for Site 4, a trend that was previously seen qualitatively in FIG. 10, panels A-C, and FIG. 13, panels A-C (and discussed in Example 8). Loess smoothed and time warped oxonium ion profiles zoomed-in for each glycosylation site can be seen in FIGS. 20 and 21 (HexNAc and sialic acid, respectively).

In its entirety, the automated similarity assessment results shown in this section illustrate the utility of using oxonium ion profiles (e.g., HexNAc and/or sialic acid oxonium ion profiles, e.g., global and/or site-specific). Targeted conventional analysis can also be used with oxonium ion profiling when a very comprehensive comparison between samples is warranted. Being that the data independent all ion fragmentation scans to generate the oxonium ion profiles can be added directly to conventional data dependent analyses, and that the results can be quantitated automatically, the additional time commitment to produce these fingerprint comparisons is quite low.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of manufacture, comprising:
providing or obtaining a batch of a test glycoprotein drug substance;
using mass spectrometry to acquire a test oxonium ion profile from a sample of the test glycoprotein drug substance batch;
comparing the test oxonium ion profile to a corresponding target oxonium ion profile of a target glycoprotein drug product; and
processing the batch of the test glycoprotein drug substance as a drug product if the difference between the test oxonium ion profile and the corresponding target oxonium ion profile is tolerable; or
taking an alternative action if the difference between the test oxonium ion profile and the target oxonium ion profile is not tolerable.

2. The method of claim 1, wherein the test glycoprotein drug substance comprises a glycoprotein that has an amino acid sequence that is identical to a glycoprotein of the target glycoprotein drug product.

3. The method of claim 1 or 2, wherein the test glycoprotein drug substance comprises an Fc fusion protein or an antibody.

4. The method of any one of the preceding claims, wherein the target glycoprotein drug product is approved under a primary approval process.

5. The method of any one of the preceding claims, wherein the target glycoprotein drug product is approved under a BLA.

6. The method of any one of the preceding claims, wherein using mass spectrometry comprises digesting the sample to produce a plurality of glycopeptides and/or glycans.

7. The method of any one of the preceding claims, wherein using mass spectrometry comprises performing liquid chromatography-tandem mass spectrometry.

8. The method of any one of the preceding claims, wherein using mass spectrometry comprises performing data-independent mass spectrometry.

9. The method of any one of the preceding claims, wherein using mass spectrometry comprises performing all ion fragmentation.

10. The method of any one of the preceding claims, wherein the test oxonium ion profile comprises one or more MS signals associated with levels of oxonium ion-containing fragments that include the following oxonium ions:
(i) HexNAc internal fragment;
(ii) Hex;
(iii) HexNAc;
(iv) sialic acid;
(v) sialic acid+H₂O;
(vi) Hex+HexNAc; or
(vii) combinations thereof.

11. The method of any one of the preceding claims, wherein the test oxonium ion profile comprises one or more MS signals associated with levels of oxonium ion-containing fragments that include oxonium ions having the following m/z values as well as any associated isotope peaks:
(i) m/z 138.06 (monoisotopic), 139.06 (+1 isotope), 140.06 (+2 isotope);
(ii) m/z 163.06 (monoisotopic), 164.06 (+1 isotope), 165.06 (+2 isotope);
(iii) m/z 204.09 (monoisotopic), 205.09 (+1 isotope), 206.09 (+2 isotope);
(iv) m/z 274.09 (monoisotopic), 275.10 (+1 isotope), 276.10 (+2 isotope);
(v) m/z 292.10 (monoisotopic), 293.11 (+1 isotope), 294.11 (+2 isotope);
(vi) m/z 366.14 (monoisotopic), 367.14 (+1 isotope), 368.14 (+2 isotope); or
(vii) combinations thereof.

12. The method of any one of the preceding claims, further comprising producing a representation of the comparison of the test oxonium ion profile to the target oxonium ion profile.

13. The method of any one the preceding claims, further comprising using mass spectrometry to acquire a target oxonium ion profile of the target glycoprotein drug product.

14. The method of any one of the preceding claims, wherein the difference between the test oxonium ion profile and the corresponding target oxonium ion profile is tolerable if the difference between a level of at least one oxonium ion-containing fragment derived from the test oxonium ion profile and a level of at least one oxonium ion-containing fragment derived from the corresponding target oxonium ion profile is less than a predetermined value.

15. The method of any one of claims 1-13, wherein the difference between the test oxonium ion profile and the corresponding target oxonium ion profile is not tolerable if the difference between a level of at least one oxonium ion-containing fragment derived from the test oxonium ion profile and a level of at least one oxonium ion-containing fragment derived from the corresponding target oxonium ion profile is greater than a predetermined value.

16. The method of any one of claims 1-13, wherein the test oxonium ion profile comprises one or more MS signals that are each associated with a level of an oxonium ion-containing fragment and the target oxonium ion profile comprises one or more MS signals that are each associated with a level of an oxonium ion-containing fragment.

17. The method of claim 16, wherein the difference between the test oxonium ion profile and the corresponding target oxonium ion profile is tolerable if the difference between 2, 3, 4, 5, or more MS signals of the test oxonium ion profile and 2, 3, 4, 5 or more corresponding MS signals of the target oxonium ion profile is each less than a predetermined value.

18. The method of claim 16, wherein the difference between the test oxonium ion profile and the corresponding target oxonium ion profile is not tolerable if the difference between 1, 2, 3, 4, 5, or more MS signals of the test oxonium ion profile and 1, 2, 3, 4, 5 or more corresponding MS signals of the target oxonium ion profile is each greater than a predetermined value.

19. The method of any one of claims 14-15 and 17-18, wherein the predetermined value is equivalent to the variability in oxonium ion profiles determined for three or more distinct batches of the target glycoprotein.

20. The method of any one of claims 14-15 and 17-18, wherein the predetermined value is 20%, 15%, 10% or 5%.

21. The method of any one of the preceding claims, wherein the alternative action comprises disposing of the batch of the test glycoprotein drug substance, classifying for disposal the batch of the test glycoprotein drug substance, labeling the batch of the test glycoprotein drug substance for disposal, reprocessing the batch of the test glycoprotein drug substance or a combination thereof.

22. The method of any one of the preceding claims, wherein the processing step comprises:
(i) formulating the batch of the test glycoprotein drug substance;
(ii) combining the batch of the test glycoprotein drug substance with a second component, e.g., an excipient or buffer;
(iii) changing the concentration of the batch of the test glycoprotein drug substance in the drug product;
(iv) lyophilizing the batch of the test glycoprotein drug substance;
(v) combining a first and second aliquot of the batch of the test glycoprotein drug substance to provide a third, larger aliquot;
(vi) dividing the batch of the test glycoprotein drug substance into smaller aliquots;
(vii) disposing the batch of the test glycoprotein drug substance into a container, e.g., a gas or liquid tight container;
(viii) packaging the batch of the test glycoprotein drug substance;
(ix) associating a container comprising the batch of the test glycoprotein drug substance with a label (e.g., labeling);
(x) shipping or moving the batch of the test glycoprotein drug substance to a different location; or
(xi) a combination thereof.
